(19) United States
(12) Patent Application Publication
(10) Pub. No.: US 2018/0237386 A1
(43) Pub. Date: Aug. 23, 2018

(54) Process For Preparation Of Vortioxetine Hydrobromide

(71) Applicant: Amneal Pharmaceuticals Company GmbH, Steinhausen (CH)

(72) Inventors: Virendra Kumar Agarwal, Ahmedabad, Gujarat (IN); Lalit Keshav Katariya, Ahmedabad, Gujarat (IN); Abhay Subodhbhai Maheta, Ahmedabad, Gujarat (IN); Rajesh Gangarambhai Rupala, Dist-Morbi, Gujarat (IN); Pankaj Chaganbhai Butani, Dist: Rajkot, Gujarat (IN); Parag Vrujlal Ajudia, Dist-Jamnagar, Gujarat (IN); Chirag Mansukhbhai Jethva, Nadiad, Gujarat (IN); Hemant Atulbhai Patel, Ahmedabad, Gujarat (IN); Viral Arvindbhai Doshi, Morbi, Gujarat (IN)

(21) Appl. No.: 15/752,082

(22) PCT Filed: Aug. 18, 2016
(86) PCT No.: PCT/EP2016/069648

§ 371 Date: Feb. 12, 2018

(30) Foreign Application Priority Data
Aug. 19, 2015 (IN) 3152/MUM/2015
Oct. 19, 2015 (IN) 3956/MUM/2015

(51) Int. Cl. C07C 319/20; C07C 323/37; C07C 323/40

(52) U.S. Cl. CPC C07C 319/20; C07C 323/40; C07C 323/37

(57) ABSTRACT

The present invention provides a process for preparation of Vortioxetine hydrobromide (I). The present invention also relates to the novel intermediate and its use in preparation of vortioxetine hydrobromide (I).

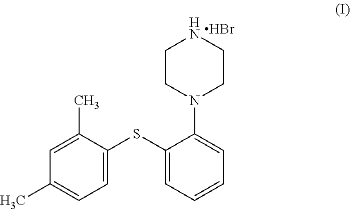 [SMILES: Br ; Cc1ccc(Sc2ccccc2N2CCNCC2)c(C)c1]

### FIELD OF INVENTION

The present invention relates to a process for preparation 1-[2-(2,4-Dimethyl-phenylsulfanyl)-phenyl]-piperazine, hydrobromide (vortioxetine hydrobromide). The present invention also relates to the novel intermediate and its use in preparation of vortioxetine hydrobromide (I).

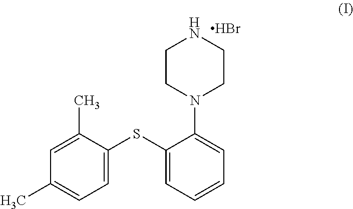 [SMILES: Br ; Cc1ccc(Sc2ccccc2N2CCNCC2)c(C)c1]

### BACKGROUND OF INVENTION

Vortioxetine hydrobromide is used as an antidepressant. It is indicated for the treatment of major depressive disorder (MDD). Vortioxetine hydrobromide is marketed in USA by Takeda Pharms under trade name Brintellix® in the form of an oral tablet.

U.S. Pat. No. 7,144,884 first disclosed vortioxetine and related compounds. The patent also discloses process for preparation of vortioxetine which involves polystyrene bound amine intermediate and resin bound complex reagents wherein reaction takes place on the resin surface. The patent disclosure is limited by the use of expensive polymer support material, slow rate of reaction and low yield making the process unsuitable for large scale industrial manufacturing. This patent also discloses other general methods for preparation including following reaction scheme I and scheme II.

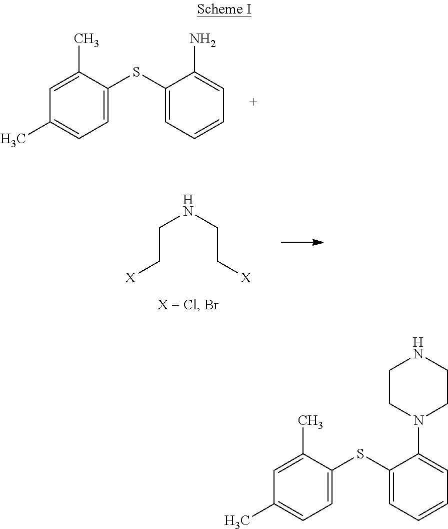

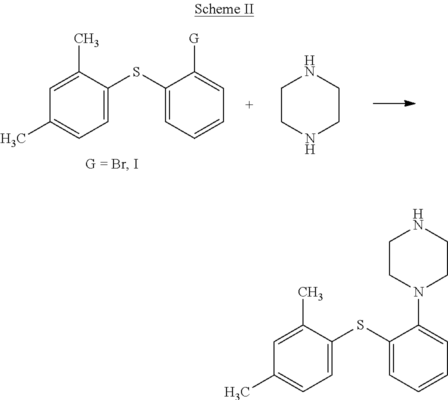

In scheme II cyclization requires a high temperature of about 200° C. Scheme III may result in a dimer impurity wherein another molecule of halo intermediate is attached to the product. US 20140256943 A1 disclose the process for preparation of vortioxetine as shown in scheme III which involves reacting compound (x), (y) and (z) in presence of a solvent, base, palladium catalyst and phosphine ligands.

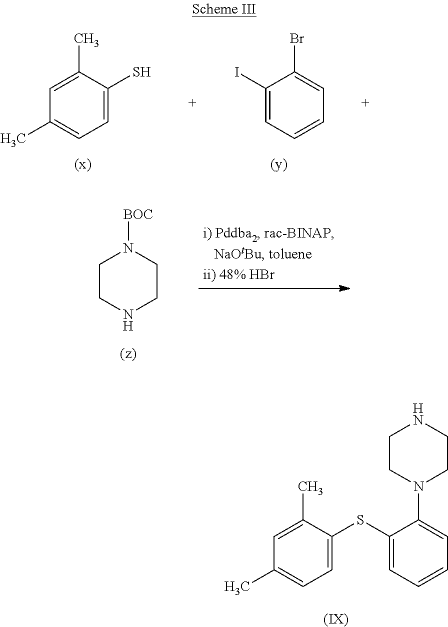

WO 2013102573 A1 discloses the process for preparation of vortioxetine hydrobromide (I) as shown in scheme IV which involves reacting compound (d), (e) and (f) in presence of a solvent, base, palladium catalyst and phosphine ligands.

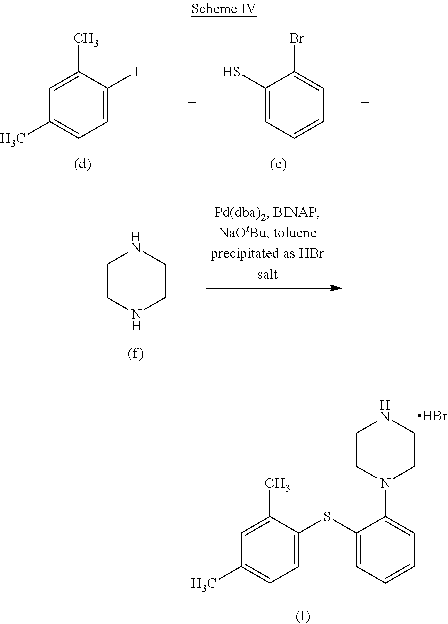

The process of US '943 and US '573 applications results in a side reaction and an overreaction due to multiple reaction sites available which produces many impurities.

WO2014191548 discloses a process for preparation of vortioxetine hydrobromide which uses sulfone or sulfoxide intermediates which is reduced to get vortioxetine as shown in scheme V.

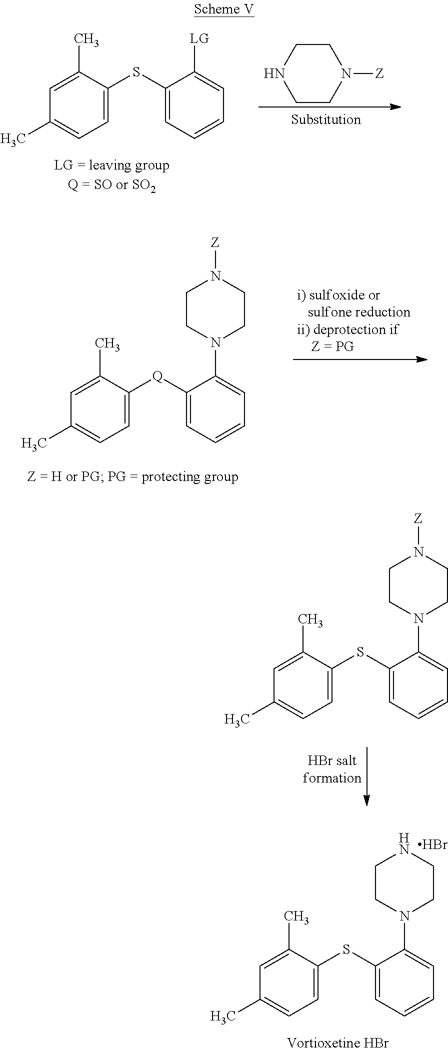

However this process results in low to moderate yield rendering it unsuitable for manufacturing on an industrial scale.

In view of the above mentioned disadvantages of prior art processes, there is a need to develop an improved process for the preparation of vortioxetine hydrobromide, which is industrially feasible and should avoid formation of dimeric and other process related impurities, while providing desired product in high yield and purity.

### SUMMARY OF THE INVENTION

The present invention provides a process for preparation of vortioxetine hydrobromide. It also provides novel intermediate of the route.

In first aspect, the present invention provides a process for preparation of vortioxetine hydrobromide (I) or its solvate.

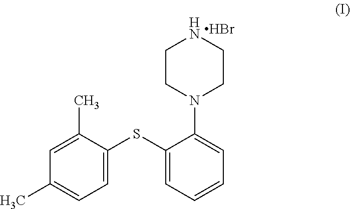 [SMILES: Br ; Cc1ccc(Sc2ccccc2N2CCNCC2)c(C)c1]

which comprises,
a) reacting 2-(2,4-dimethyl-phenylsulfanyl)-phenylamine (II) or its salt

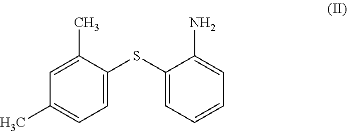 [SMILES: Cc1ccc(Sc2ccccc2N)c(C)c1]

with haloacetyl halide (III)

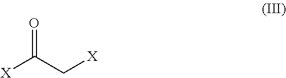

to give 2-halo-N-[2-(2,4-dimethyl-phenylsulfanyl)-phenyl]-acetamide (IV);

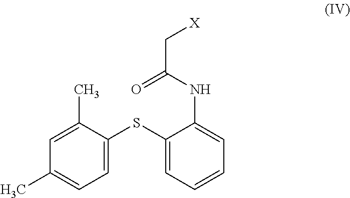

wherein X is halogen
b) reacting 2-halo-N-[2-(2,4-dimethyl-phenylsulfanyl)-phenyl]-acetamide (IV) with ethanolamine or its salt to give N-[2-(2,4-dimethyl-phenylsulfanyl)-phenyl]-2-(2-hydroxy-ethylamino)-acetamide (V) or its salt;

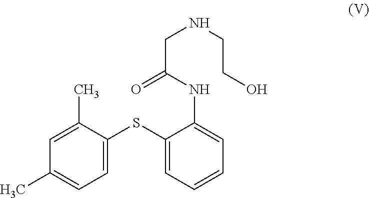 [SMILES: Cc1ccc(Sc2ccccc2NC(=O)CNCCO)c(C)c1]

c) reacting N-[2-(2,4-dimethyl-phenylsulfanyl)-phenyl]-2-(2-hydroxy-ethylamino)-acetamide (V) or its salt with protecting reagent to give compound of formula (VI);

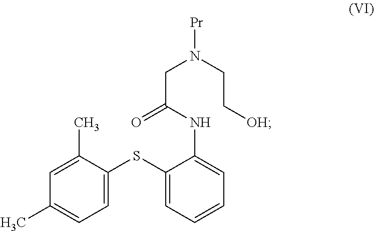

Wherein Pr is protecting group
d) cyclizing compound of formula (VI) to give compound of formula (VII);

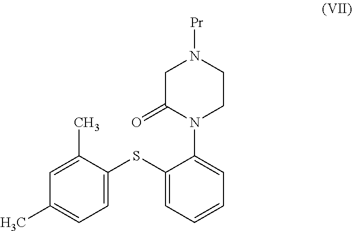

e) deprotecting group Pr from compound (VII) to give 1-[2-(2,4-dimethyl-phenylsulfanyl)-phenyl]-piperazin-2-one (VIII) or its salt;

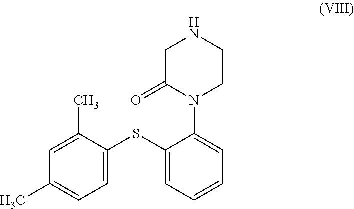 [SMILES: Cc1ccc(Sc2ccccc2N2CCNCC2=O)c(C)c1]

f) reducing compound of formula (VIII) or its salt to give vortioxetine (IX) or its salt;

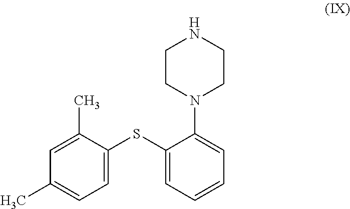 [SMILES: Cc1ccc(Sc2ccccc2N2CCNCC2)c(C)c1]

and optionally converting vortioxetine (IX) or its salt to vortioxetine hydrobromide (I) or its solvate.

In yet another aspect, step (e) and (f) can optionally be interchangeable in their sequence for preparation of vortioxetine (IX) or its salt.

In yet another aspect, the present invention provide the process for the preparation of vortioxetine (IX) which comprises,
a) reacting N-[2-(2,4-dimethyl-phenylsulfanyl)-phenyl]-2-(2-hydroxy-ethylamino)-acetamide (V) or its salt with protecting reagent to give compound of formula (VIa);

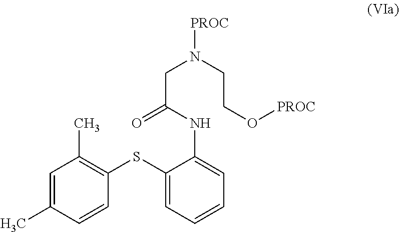

b) cyclizing compound of formula (VIa) to give compound of formula (VII);

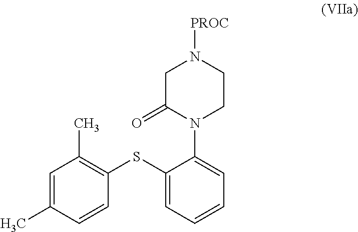

where in PROC is a protecting agent chosen such that it act as protecting group at nitrogen and leaving group when attached with oxygen under cyclization condition.

In another aspect, the present invention provides a process for preparation of vortioxetine(IX) or its salt comprising reducing compound of formula (VIII) or its salt.

In second aspect, the present invention provides a process for preparation of vortioxetine hydrobromide (I) or its solvate

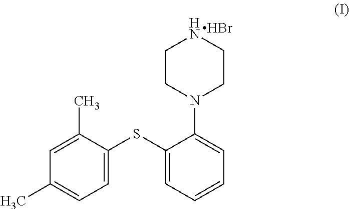 [SMILES: Br ; Cc1ccc(Sc2ccccc2N2CCNCC2)c(C)c1]

which comprises,
a) reacting 2-(2,4-dimethyl-phenylsulfanyl)-phenylamine (II) or its salt

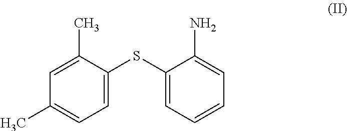 [SMILES: Cc1ccc(Sc2ccccc2N)c(C)c1]

with haloacetyl halide (III)

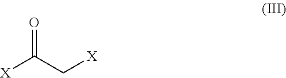

to give 2-halo-N-[2-(2,4-dimethyl-phenylsulfanyl)-phenyl]-acetamide (IV),

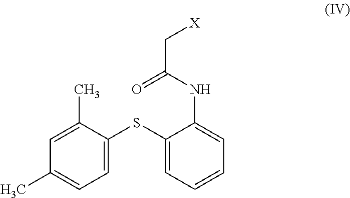

wherein X is halogen
b) reacting 2-halo-N-[2-(2,4-dimethyl-phenylsulfanyl)-phenyl]-acetamide (IV) with ethanolamine or its salt to give N-[2-(2,4-Dimethyl-phenylsulfanyl)-phenyl]-2-(2-hydroxy-ethylamino)-acetamide (V) or its salt,

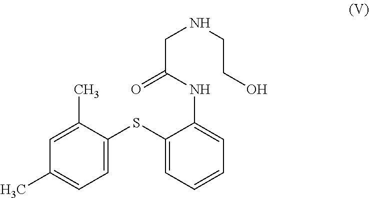 [SMILES: Cc1ccc(Sc2ccccc2NC(=O)CNCCO)c(C)c1]

c) cyclizing compound of formula (V) to give 1-[2-(2,4-dimethyl-phenylsulfanyl)-phenyl]-piperazin-2-one (VIII) or its salt,

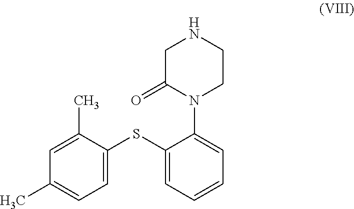 [SMILES: Cc1ccc(Sc2ccccc2N2CCNCC2=O)c(C)c1]

d) reducing compound of formula (VIII) to give vortioxetine (IX) or its salt, and

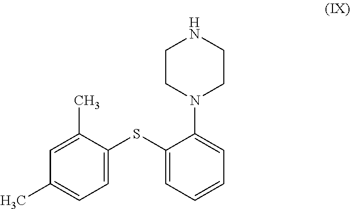 [SMILES: Cc1ccc(Sc2ccccc2N2CCNCC2)c(C)c1]

optionally converting vortioxetine (IX) or its salt to vortioxetine hydrobromide (I) or its solvate

In third aspect, the present invention provides a process for preparation of vortioxetine hydrobromide salt (I)

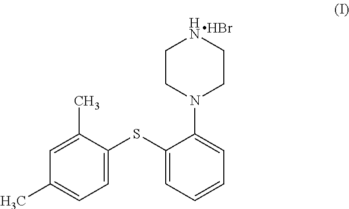 [SMILES: Br ; Cc1ccc(Sc2ccccc2N2CCNCC2)c(C)c1]

which comprises,
a) reacting 2-(2,4-dimethyl-phenylsulfanyl)-phenylamine (II) or its salt

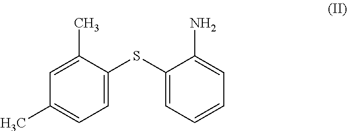 [SMILES: Cc1ccc(Sc2ccccc2N)c(C)c1]

with 2-halo-N-(2-hydroxyethy)-acetamide (X)

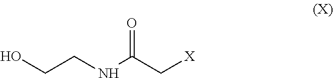

wherein X is halogen
to give 2-[2-(2,4-Dimethyl-phenylsulfanyl)-phenylamino]-N-(2-hydroxy-ethyl)-acetamide (XI) or its salt;

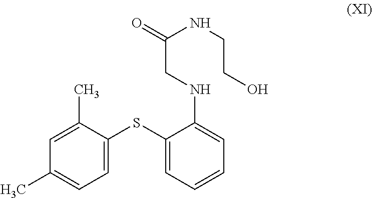 [SMILES: Cc1ccc(Sc2ccccc2NCC(=O)NCCO)c(C)c1]

b) cyclizing compound of formula (XI) to give 4-[2-(2,4-dimethyl-phenylsulfanyl)-phenyl]-piperazin-2-one (XII) or its salt,

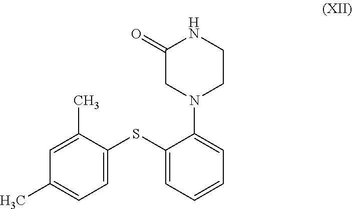 [SMILES: Cc1ccc(Sc2ccccc2N2CCNC(=O)C2)c(C)c1]

c) reducing compound of formula (XII) to give vortioxetine (IX), and

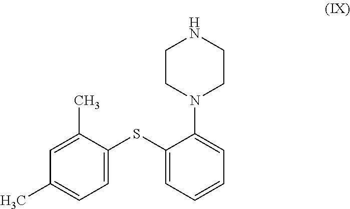 [SMILES: Cc1ccc(Sc2ccccc2N2CCNCC2)c(C)c1]

optionally converting it to vortioxetine hydrobromide (I)

In fourth aspect, the present invention provides a process for preparation of vortioxetine hydrobromide (I) or its solvate

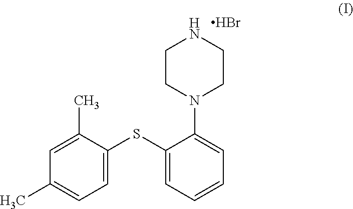 [SMILES: Br ; Cc1ccc(Sc2ccccc2N2CCNCC2)c(C)c1]

which comprises,
a) reacting 1-halo-2,4-dimethylbenzene (XIII)

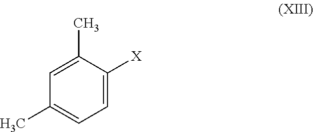

with 2-halobenzenethiol (XIV)

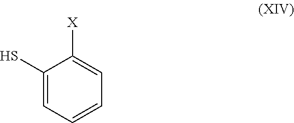

to give 1-(2-halo-phenylsulfanyl)-2,4-dimethyl-benzene (XV);

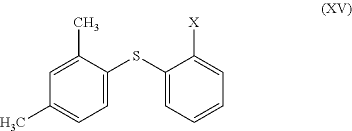

wherein X is halogen
b) reacting 1-(2-halo-phenylsulfanyl)-2,4-dimethyl-benzene (XV) with piperazine 2-one (XVI)

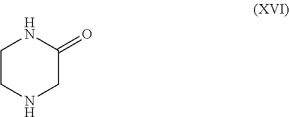

to give 4-[2-(2,4-Dimethyl-phenylsulfanyl)-phenyl]-piperazin-2-one (XII);

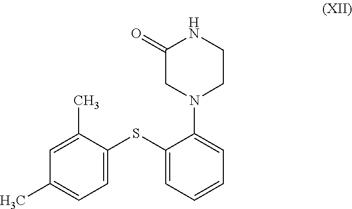 [SMILES: Cc1ccc(Sc2ccccc2N2CCNC(=O)C2)c(C)c1]

c) reducing 4-[2-(2,4-Dimethyl-phenylsulfanyl)-phenyl]-piperazin-2-one (XII) to vortioxetine (IX) and

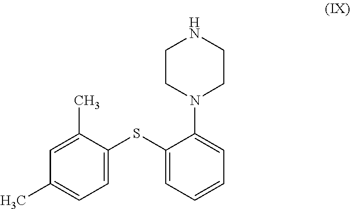 [SMILES: Cc1ccc(Sc2ccccc2N2CCNCC2)c(C)c1]

d) optionally converting vortioxetine (IX) to vortioxetine hydrobromide (I).

In another aspect, the present invention provides a process for preparation of vortioxetine hydrobromide (I) comprising a step of reducing 4-[2-(2,4-Dimethyl-phenylsulfanyl)-phenyl]-piperazin-2-one (XII).

In yet another aspect, the present invention provides novel intermediate of following formulas or salt thereof.

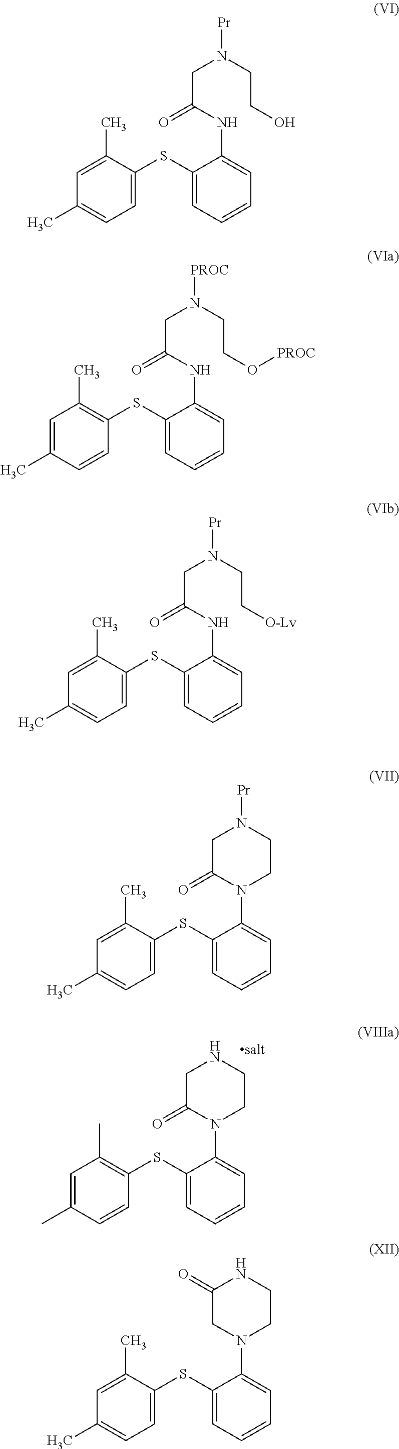

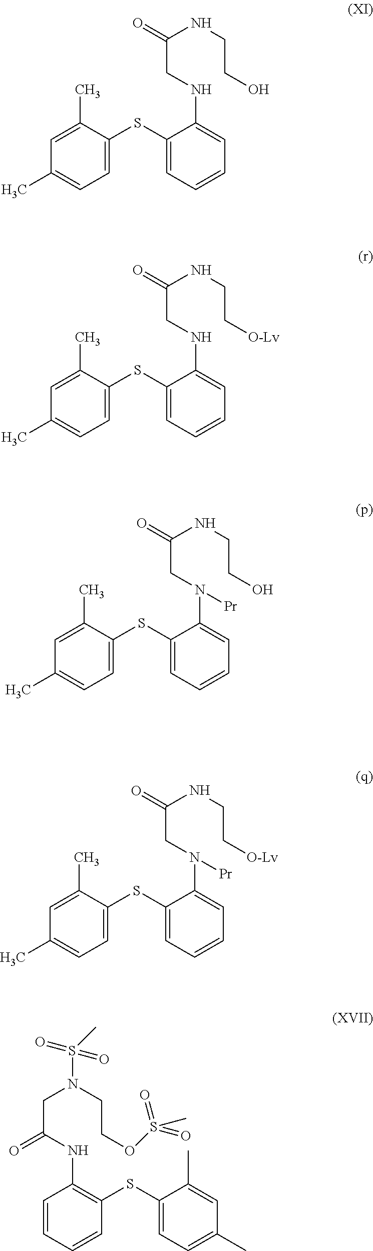

wherein X is halogen; Pr is protecting group; Lv is leaving group selected from mesyl, tosyl, nosyl; salt is organic or inorganic salt; PROC is a protecting agent such that it act as protecting group at nitrogen and leaving group when attached to oxygen under cyclization condition.

### DETAIL DESCRIPTION OF INVENTION

As used herein, the term “leaving group” can be defined as part of a substrate that cleaved by the action of a nucleophile.

As used herein, the term “halogen” refers to an atom selected from the group consisting of F, Cl, Br and I.

As referred here in, base used in any reaction step of present invention is selected from any kind of following base as single or in any combination of mixture or in aqueous form depending upon the kind and nature of the reaction. Base used in the present invention can be inorganic or organic base. Inorganic base are alkoxide, hydroxide, carbonate, bicarbonate or hydride of alkali or alkaline earth metal. Inorganic base are selected from sodium tert butoxide, potassium tert butoxide, lithium methoxide, lithium ethoxide, sodium methoxide, sodium ethoxide, potassium methoxide, potassium ethoxide, sodium hydroxide, potassium hydroxide, lithium hydroxide, cesium carbonate, potassium carbonate, sodium carbonate, lithium carbonate, sodium bicarbonate, potassium bicarbonate, lithium bicarbonate, sodium amide, sodium hydride, potassium hydride, lithium hydride, potassium phosphate, sodium phosphate and the like or mixtures thereof. Organic base are selected from triethylamine (TEA), diethylamine (DEA), pyridine, 1,8-diazabicyclo[5.4.0]undec-7-ene (DBU), diisopropyl ethylamine (DIPEA) and 1,4-diazabicyclo[2.2.2]octane (DABCO), imidazole, N,N-dimethyl aniline, N,N-dimethyl amino pyridine (DMAP), 1,5-diazabicyclo[4.3.0]non-5-ene (DBN), n-butyl lithium, lithium diisopropylamide (LDA), lithium hexamethyldisilazide (LiHMDS), sodium hexamethyldisilazide (NaHMDS), potassium hexamethyldisilazide (KHMDS) and the like or mixtures thereof.

As referred here in, organic solvent used in any reaction step of present invention is selected from any kind of following solvent as single solvent or mixture of one or more solvent. The selection of solvent depends upon the nature of the reaction. Organic solvent used in the present invention is selected from chlorinated hydrocarbons such as dichloromethane, dichloroethane, chloroform or carbon tetrachloride; aromatic hydrocarbon such as toluene, xylene; ether such as dioxan, tetrahydrofuran (THF), methyl tertbutyl ether (MTBE); nitrile such as acetonitrile; ester such as ethylacetate, isopropyl acetate; ketone such as acetone, methyl isobutyl ketone (MIBK), methyl ethyl ketone (MEK); polar aprotic such as N,N-dimethylformamide (DMF), dimethyl sulfoxide (DMSO), dimethyl acetamide (DMAc), N-methylpyrrolidone (NMP); polar protic such as alcoholic solvent C₁₋₄ alcohol such as methanol, ethanol, isopropanol, propanol, butanol and the like; water or mixtures thereof.

As referred herein, the term ‘salt’ of any intermediate or vortioxetine includes pharmaceutically acceptable acid addition salts formed with organic or inorganic acids. Exemplary of such organic salts are those but not limited to maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-toulenesulfonic acid, p-aminobenzoic, glutamic, benzenesulfonic and theophylline acetic acids. Exemplary of such inorganic salts are those but not limited to hydrochloric, hydrobromic, sulfuric, sulfamic, phosphoric and nitric acids.

Accordingly, in first embodiment, the present invention provides a process for preparation of vortioxetine hydrobromide (I) or its solvate

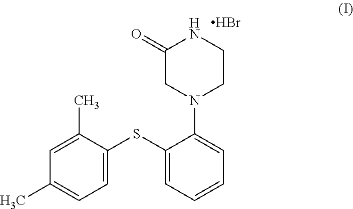 [SMILES: Br ; Cc1ccc(Sc2ccccc2N2CCNC(=O)C2)c(C)c1]

which comprises,
a) reacting 2-(2,4-dimethyl-phenylsulfanyl)-phenylamine (II) or its salt

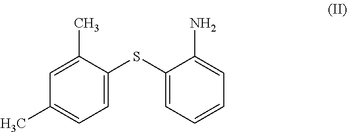 [SMILES: Cc1ccc(Sc2ccccc2N)c(C)c1]

with haloacetyl halide (III)

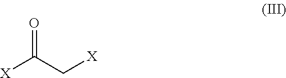

to give 2-halo-N-[2-(2,4-dimethyl-phenylsulfanyl)-phenyl]-acetamide (IV);

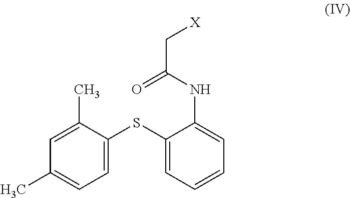

wherein X is halogen
b) reacting 2-halo-N-[2-(2,4-dimethyl-phenylsulfanyl)-phenyl]-acetamide (IV) with ethanolamine or its salt to give N-[2-(2,4-dimethyl-phenylsulfanyl)-phenyl]-2-(2-hydroxy-ethylamino)-acetamide (V) or its salt;

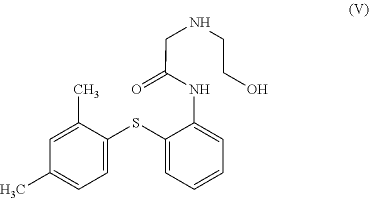 [SMILES: Cc1ccc(Sc2ccccc2NC(=O)CNCCO)c(C)c1]

c) reacting N-[2-(2,4-dimethyl-phenylsulfanyl)-phenyl]-2-(2-hydroxy-ethylamino)-acetamide (V) or its salt with protecting reagent to give compound of formula (VI);

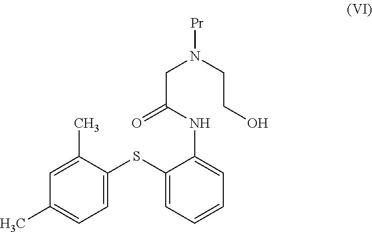

wherein Pr is protecting group
d) cyclizing compound of formula (VI) to give compound of formula (VII);

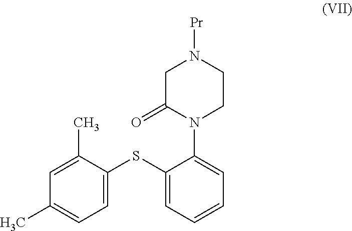

e) deprotecting Pr group from compound (VII) to give 1-[2-(2,4-Dimethyl-phenylsulfanyl)-phenyl]-piperazin-2-one (VIII) or its salt;

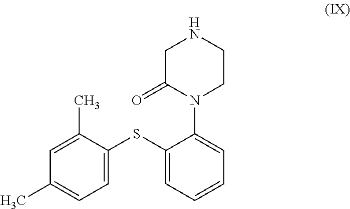 [SMILES: Cc1ccc(Sc2ccccc2N2CCNCC2=O)c(C)c1]

f) reducing compound of formula (VIII) or its salt to give vortioxetine (IX) or its salt;

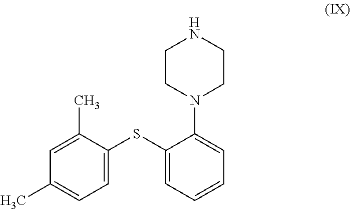 [SMILES: Cc1ccc(Sc2ccccc2N2CCNCC2)c(C)c1]

and optionally converting vortioxetine (X) or its salt to vortioxetine hydrobromide (I) or its solvate.

In step a) 2-(2,4-Dimethyl-phenylsulfanyl)-phenylamine (II) or its salt is reacted with haloacetyl halide (III) to give 2-halo-N-[2-(2,4-dimethyl-phenylsulfanyl)-phenyl]-acetamide (IV). Haloacetyl halide (III) used can be chloro acetyl chloride, bromo acetyl bromide, bromo acetyl chloride, and the like. Halogen in halo acetyl halide can be selected from fluoro, chloro, bromo or iodo. The reaction is carried out in presence of base and organic solvent. Haloacetyl chloride is taken in 1-2 mol equivalent to compound (II). Base can be selected from any organic base or inorganic base as mentioned in the beginning of detail description. Preferred base are hydroxide, carbonate, bicarbonate or hydride of alkali or alkaline earth metal. It is used in the form of an aqueous solution. For example aqueous Sodium or potassium carbonate, aqueous sodium or potassium bicarbonate may be used.

Organic solvent can be selected from any solvent or mixture thereof as mentioned in the beginning of detail description. Preferred solvent are aromatic hydrocarbons such as toluene, xylene. Other solvent such as chlorinated hydrocarbons, ether, nitrile, ester, ketone can also be used. The reaction is carried out at about 0° C. to reflux temperature, preferably at 25° C. to about 35° C. After completion of the reaction, organic phase is separated. It may be used as such for next step for ethanolamine condensation or it may be isolated.

In step b), 2-halo-N-[2-(2,4-dimethyl-phenylsulfanyl)-phenyl]-acetamide (IV) is condensed with ethanolamine or its salt to give N-[2-(2,4-Dimethyl-phenylsulfanyl)-phenyl]-2-(2-hydroxy-ethylamino)-acetamide (V) or its salt. Ethanolamine is taken in excess about 4 to 6 mol equivalent to the compound (IV). The reaction is carried out at elevated temperature 100° C. to 110° C. in presence of an organic solvent. Organic solvent can be selected from any solvent or mixture thereof as mentioned in the beginning of detail description. Preferred solvent is aromatic hydrocarbon such as toluene, xylene or ester solvent such as ethylacetate, isopropyl acetate. Other solvent from group such as chlorinated hydrocarbons, ether, nitrile, ketone can also be used. After completion of reaction, the reaction mixture is washed with water. Organic phase is separated and evaporated to give the compound (V) or it can be converted to its salt by reacting it with acid. For example hydrochloride salt can be prepared by reacting organic phase obtained after work up can be treated with aq. hydrochloric acid and heated at 40-50° C. for 2-4 h. The compound precipitate out as its hydrochloride salt which is filtered, washed with reaction solvent and then dried in oven.

In step c) N-[2-(2,4-dimethyl-phenylsulfanyl)-phenyl]-2-(2-hydroxy-ethylamino)-acetamide(V) or its salt is subsequently reacted with protecting reagent having protecting group Pr to give compound of formula (VI). Protecting group Pr for this reaction step can be selected from tertbutyloxycarbonyl (boc), triphenylmethyl (trityl), benzyloxycarbonyl (cbz), benzyl, trifluoroacetyl (COCF₃), acetyl, silyl and the like. Appropriate protecting reagent can be used to induce specific protecting group. For example boc anhydride can be used for boc protection, trityl chloride or benzyl chloride can be used for trityl or benzyl protection respectively, trifluoroacetyl chloride, trifluoro acetic anhydride or acetyl chloride used for trifluoroacetyl (COCF₃) or acetyl protection respectively. The reaction is carried out in an organic solvent in presence of base. Base can be selected from any organic base or inorganic base as mentioned in the beginning of detail description. Preferred base are organic base such as triethylamine (TEA), diethylamine (DEA), pyridine, 1,8-diazabicyclo[5.4.0]undec-7-ene (DBU), diisopropyl ethylamine (DIPEA) and 1,4-diazabicyclo[2.2.2]octane (DABCO) and the like or mixtures thereof. Organic solvent can be selected from any solvent or mixture thereof as mentioned in the beginning of detail description. The preferred organic solvent used for this step may be selected from aromatic hydrocarbon such as toluene, xylene. Other solvent from group such as chlorinated hydrocarbons, ether, nitrile, ester, ketone, polar aprotic solvent or polar protic such as C₁₋₄ alcohol can also be used.

For example, compound of formula (V) is reacted with boc-anhydride in toluene in presence of triethylamine at ambient temperature at 25° C. to 35° C. for about 6 to 10 h to give boc protected compound after customary work up and purification. Purification may be effected by crystallization methods including solvent antisolvent method. Antisolvent used can be selected from non polar solvent such as hexane, cyclohexane, heptane, pat ether and the like. For example toluene and cyclohexane as solvent and antisolvent respectively can be used for purification. In step d), Cyclization of compound of formula (VI) is carried out by preparing reactive derivative of hydroxyl group of compound (VI) i.e. formula (VIb), which in turn is cyclized to give compound of formula (VII). The cyclization can be carried out by converting hydroxyl group of compound (VI) to o-mesyl, o-tosyl or o-nosyl group which is good leaving group designated as ‘Lv’ as shown in Scheme VI and then cyclization of compound (VIb) gives compound (VII) in appropriate reaction condition.

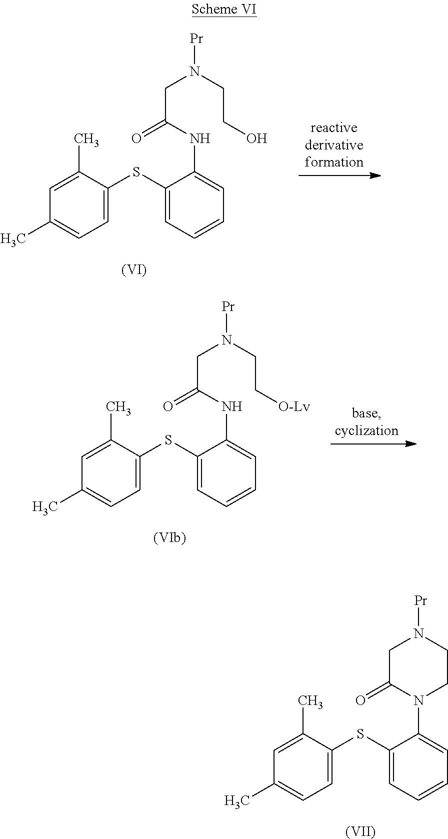

Wherein Pr and Lv designates protecting group and leaving group respectively.

In this process, compound of formula (VI) can be converted to its reactive derivative compound (VIb) by doing sulfonylation at hydroxyl group. Various sulfonylating reagents can be used such as methane sulfonyl chloride (mesyl chloride), p-toluene sulfonyl chloride (tosyl chloride) or 4-Nitrobenzenesulfonyl chloride (nosyl chloride) to prepare reactive derivative of formula (VIb) wherein Lv is mesyl, tosyl or nosyl obtained according to the sulfonylating reagent used. Sulfonylating reagent is used in 1.4 to 2.5 mol equivalent to compound (VI).

The reaction can be carried out at ambient temperature, preferably at low temperature from 0° C. to 10° C. in the presence of base and an organic solvent. Preferred bases are organic base such as triethylamine (TEA), diethylamine (DEA), pyridine, 1,8-diazabicyclo[5.4.0]undec-7-ene (DBU) and 1,4-diazabicyclo[2.2.2]octane (DABCO) and the like or mixtures thereof. Inorganic base can be selected from hydroxide, carbonate, bicarbonate of alkali and alkaline earth metal. An organic solvent can be selected from any solvent or mixture thereof as mentioned in the beginning of detail description. Preferred organic solvent is aromatic hydrocarbon such as toluene, xylene and the like or mixture thereof. Other solvent from group such as chlorinated hydrocarbons, ether, nitrile, ester, and ketone can also be used. After completion of reaction, the mixture is washed with brine. The organic phase is separated and used as such insitu for cyclization.

The above obtained compound (VIb) can be cyclized to compound (VII) in presence of base in organic solvent. Base can be selected from any organic base or inorganic base as mentioned above. Preferred base are inorganic base selected from group of alkoxide, hydroxide, carbonate, bicarbonate or hydride of alkali or alkaline earth metal. Preferred base is selected from potassium carbonate, sodium carbonate, sodium bicarbonate, potassium bicarbonate, sodium hydroxide, potassium hydroxide, sodium tert butoxide, potassium tert butoxide, sodium ethoxide, potassium ethoxide, sodium methoxide and the like or mixtures thereof. Organic solvent can be selected from any solvent or mixture thereof as mentioned in the beginning of detail description. Preferred organic solvent used for cyclization is aromatic hydrocarbon such as toluene, xylene and the like. Other solvent from group such as polar protic solvent alcohol, polar aprotic solvents, chlorinated hydrocarbons, ether, nitrile, ester, and ketone can also be used. After completion of the reaction, the reaction mixture is quenched with water and extracted. Organic phase is separated and evaporated. The product is isolated by adding a non polar solvent such cyclohexane, heptane, hexane, pat ether to the residue.

In step e), deprotection of compound (VIII) can be carried out by removing protecting group Pr which gives 1-[2-(2,4-Dimethyl-phenylsulfanyl)-phenyl]-piperazin-2-one (IX) or its salt. The step of deprotection can be carried out via acid, base or hydrogenolysis, depending upon the protecting group Pr. If protecting group Pr is boc or trityl, then it can be deprotected by acid. If protecting group Pr is benzyl or cbz, then it can be removed by hydrogenation. Pretection, deprotection reaction is well described in the literature. Deprotection can be carried out in an organic solvent selected from any solvent or mixture thereof as mentioned in the beginning of detail description. Preferred solvent can be selected from ester such as ethyl acetate, isopropyl acetate. Other solvent from group such as polar protic solvent alcohol, chlorinated hydrocarbons, aromatic hydrocarbon, ether, nitrile, ketone, polar aprotic solvent, water or mixture of solvent with water can also be used. Acid used for deprotection step may be organic or mineral acid. Further it can be either aqueous solution, concentrated solution or saturated in organic solvent such as hydrochloric acid in isopropanol (IPA-HCl), hydrobromic acid in acetic acid (HBr in AcOH).

Moreover, deprotection with acid simultaneously deprotects and forms salt as an end product in a single step which has advantage of ease in purification and isolation as compared to base as an end product from reaction mixture. Upon deprotection of compound (VIII) where protecting group Pr is boc gives hydrochloride salt of 1-[2-(2,4-Dimethyl-phenylsulfanyl)-phenyl]-piperazin-2-one (XI). The salt obtained after deprotection can be organic or inorganic depends on the acid used for the deprotection. Preferred salt of 1-[2-(2,4-Dimethyl-phenylsulfanyl)-phenyl]-piperazin-2-one (IX) can be selected from a group of hydrochloric acid (HCl), hydrobromic acid (HBr), sulfuric acid, phosphoric acid, nitric acid, methane sulfonic acid, p-toluene sulfonic acid (PTSA), trifluorosulfonic acid (TFA) and the like. Respective acid can be used for deprotection to get the salt of that acid. The product in its salt form is isolated by filtration.

In step f), reduction of 1-[2-(2,4-Dimethyl-phenylsulfanyl)-phenyl]-piperazin-2-one (IX) or its salt gives vortioxtine (IX) or its salt (X). The step of reduction can be carried out using reducing reagents such as lithium aluminum hydride (LiAlH₄), borane-dimethyl sulfide (Borane-DMS), combination of borohydride and lewis acid such as sodium borohydride (NaBH₄) and BF₃-etherate. Other lewis acids used are ZnCl₂, AlCl₃, MgCl₂, BF₃, TiCl₄ and the like. Combination of borohydride with other reagent such as acetic acid, pyridine, POCl₃, trimethylsilyl is also used as reducing reagent. Borohydride is selected from NaBH₄, lithium borohydride (LiBH₄), sodium cyanoborohydride (NaCNBH₃). Sodium bis(2-methoxyethoxy) aluminumhydride (trade names Red-Al or vitride) is also used as reducing reagent. Organic solvent used for reduction process can be selected from any solvent or mixture thereof as mentioned in the beginning of detail description. Solvents used for reduction process is selected from C₁₋₄alcohol such as methanol, ethanol, propanol, isopropanol, butanol; ether such as THF, dioxan, methyl tertbutyl ether (MTBE); nitrile such as acetonitrile; aromatic hydrocarbon such as toluene, xylene and the like; chlorinated solvent such as dichloromethane, dichloroethane, chloroform or carbon tetrachloride or mixtures thereof.

The reaction condition depends upon selection of reducing reagent taken. The reaction temperature varies from −10° C. to reflux temperature depending upon the reagent chosen. LiAlH₄, borohydride, and combination of borohydride with other reagent require low temperature around −10° C. to room temperature, whereas a reagent such as vitride requires room temperature to reflux temperature. The reaction work up is usually performed with acid to break the complex formed during reaction. For example in case of borane-DMS complex, the reaction is carried out at 25° to 45° C. to reflux for 4-8 h. The work up includes quenching of the reaction with dil. acid solution such as aq. HCl solution with heating at 35° to 45° C. for about 2-3 h. The reaction mixture is cooled and basified with aq. NaOH solution till basic pH preferably 8.5 to 10.5. The reaction mixture is extracted and evaporated. The residue is dissolved in 2-butanol or tertiary-butanol. Aq. hydrobromic acid is added to it and heated to 60-70° C. for about 1 h. The precipitated solid is filtered and dried to give vortioxetine hydrobromide (I) or its solvate. The type of solvate depends upon the solvent taken for salt formation.

In yet another embodiment, step (e) and (f) can optionally be interchangeable in their sequence for preparation of vortioxetine (IX) or its salt. Compound (VII) is reduced first to give boc protected vortioxetine (VIIb) which is then deprotected to give vortioxetine (IX). In yet another aspect, the present invention provide the process for the preparation of vortioxetine (IX) which comprises,
a) reacting N-[2-(2,4-dimethyl-phenylsulfanyl)-phenyl]-2-(2-hydroxy-ethylamino)-acetamide (V) or its salt with protecting reagent to give compound of formula (VIa);

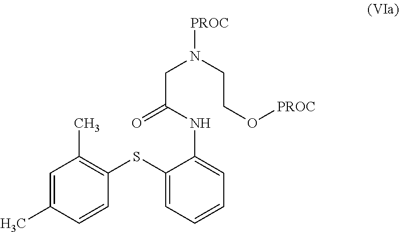

b) cyclizing compound of formula (VIa) to give compound of formula (VII);

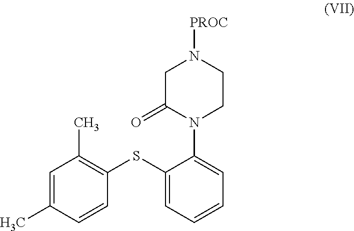

where in PROC is a protecting group.

In this process, protecting group (PROC) is so chosen that it act as protecting group at nitrogen atom and leaving group at oxygen atom when subjected to cyclization condition. The PROC can be selected from methane sulfonyl, p-toluene sulfonyl, nosyl, COCF₃(trifluro acetyl), acetyl, acyl, benzyl, substituted benzyl, benzoyl, trimethylsilyl, tert butyl dimethyl sily (TBDMS), trifluoromethylsulfonate (OTf) and the like. Protecting reagent is selected with a view that it should be capable of doing protection at both site nitrogen atom and oxygen atom.

Protecting reagent can be selected from sulfonylating reagents such as methane sulphonyl chloride (mesyl chloride), tosyl chloride or nosyl chloride; acylating reagents such as trifluoromethyl carbonyl chloride, acyl chloride or acyl anhydride, acetyl chloride, acetic anhydride, trifluoroacetic anhydride; other reagents such as benzoyl chloride, trifluoromethyl sulfonate reagents and the like. The reagent is generally used in 2.0 mol or greater than 2.0 mol equivalent to compound (VII), preferably 2-4 mol equivalent. Protection reaction is carried out in presence of base and solvent. Base can be selected from any organic base or inorganic base as mentioned in the beginning of detail description. Preferred base are organic base such as triethylamine (TEA), diethylamine (DEA), pyridine, 1,8-diazabicyclo[5.4.0]undec-7-ene (DBU) and 1,4-diazabicyclo[2.2.2]octane (DABCO) and the like or mixtures thereof. Inorganic base are hydroxide, carbonate, bicarbonate, alkoxide, hydride of alkali and alkaline earth metal. Preferred organic solvent is chlorinated hydrocarbons such as dichloromethane, dichloroethane, chloroform or carbon tetrachloride or mixture thereof. Other solvent from group such as aromatic hydrocarbon, ether, nitrile, ester, ketone can also be used.

Compound (VIa) under cyclization condition is converted to compound (VIIa) by heating the solution of compound (VIa) in organic solvent with base. Under such condition protecting group on oxygen act as leaving group while protecting group on nitrogen act as protecting group. Base can be selected from any organic base or inorganic base. Preferred base are inorganic base from group alkoxide, hydroxide, carbonate, bicarbonate or hydride of alkali or alkaline earth metal. Preferred base is selected from potassium carbonate, sodium carbonate, sodium bicarbonate, potassium bicarbonate, sodium hydroxide, potassium hydroxide, sodium tert butoxide, potassium tert butoxide, sodium ethoxide, potassium ethoxide, sodium methoxide and the like or mixtures thereof. Organic solvent can be selected from any solvent or mixture thereof as mentioned in the beginning of detail description. Preferred organic solvent used for cyclization is aromatic hydrocarbon such as toluene, xylene or polar aprotic solvent such as DMF, DMSO, DMAc, NMP or mixtures thereof. Other solvent from group such as polar protic solvent alcohol, chlorinated hydrocarbons, ether, nitrile, ester, ketone can also be used. The cyclization reaction can be carried out 50-60° C. for about 10 to 12 hours. Customary work up like quenching in water and extracting in solvent and evaporating gives compound of formula (VII).

In another embodiment, the present invention provides a process for preparation of Vortioxetine (IX) which comprises
(a) deprotecting group Pr from compound (VII) with acid to give salt of 1-[2-(2,4-dimethyl-phenylsulfanyl)-phenyl]-piperazin-2-one (VIIIa);

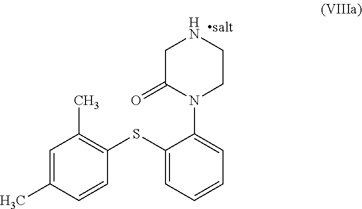

where in Pr is protecting group
(b) reducing salt of formula (VIIIa) to give salt of vortioxetine (IXa);

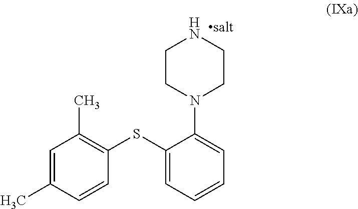

and optionally converting salt of vortioxetine (IXa) to vortioxetine hydrobromide (I) or its solvate.

Starting compound 2-(2,4-Dimethyl-phenylsulfanyl)-phenylamine (II) can be prepared by the methods known in the art. 2,4-dimethylbenzenethiol (A) is condensed with 1-Chloro-2-nitro-benzene (B) in presence of base in organic solvent. Base can be selected from any organic base or inorganic base as mentioned in the beginning of detail description. Preferred base are selected from alkoxide, hydroxide, carbonate, bicarbonate or hydride of alkali or alkaline earth metal. The example of preferred base sodium hydroxide, potassium hydroxide, lithium hydroxide, cesium carbonate, potassium carbonate, sodium carbonate, sodium bicarbonate, potassium bicarbonate and the like or mixtures thereof. Preferably an aqueous solution of inorganic base is used. Organic solvent used can be selected from any solvent or mixture thereof as mentioned in the beginning of detail description. Preferred solvents are polar protic solvent or its mixture with water. Polar protic solvent are C₁₋₄ alcohol such as methanol, ethanol, isopropanol, propanol, butanol and the like or mixture thereof. Other solvent from group such as chlorinated hydrocarbons, aromatic hydrocarbon, ether, nitrile, ester, ketone, polar aprotic solvent can also be used such as THF, acetonitrile, water or mixture thereof. The reaction is carried out at elevated temperature at about 50° C. to about 90° C. or to reflux for about 4 to 14 hours. The compound 2,4-Dimethyl-1-(2-nitro-phenylsulfanyl)-benzene (C) obtained as yellowish crystalline solid can be used for next step without further purification.

2,4-Dimethyl-1-(2-nitro-phenylsulfanyl)-benzene (C) as obtained above is reduced to 2-(2,4-Dimethyl-phenylsulfanyl)-phenylamine (II). Reduction of nitro to amino group can be performed by various methods. Reduction can be done by adding acid over transition metal. Transition metal used is Fe, Sn, Zn, Mn, Mg. Acid used can be mineral acid such as HCl, sulfuric acid, nitric acid, phosphoric acid or organic acid such as AcOH, formic acid. For example Fe/HCl, Sn/HCl, Fe/AcOH, Zn/NH₄Cl. Other methods include hydrogenation using raney Ni or over noble metal such as Pt, Pd, Rh, Ru either supported on carbon or in its complex form. Other method includes use of Ammonium formate/Pd, Pd/ammonium formate/formic acid, dithionate. The reaction condition of reduction varies and depends on the method of reduction chosen. For example, reduction is done using AcOH and Fe powder or Zinc dust and ammonium chloride, conducted at elevated temperature at 60-100° C. After completion of the reaction, the reaction mixture is filtered through hyflo bed to remove salt. The hyflo bed is washed with solvent such as methanol. Combined filtrate is evaporated up to thick residue which is dissolved in ethylacetate and washed with water to give crude 2-(2,4-Dimethyl-phenylsulfanyl)-phenylamine (II) which can be converted to its acid salt. Acid salt can be hydrochloride, hydrobromide, hydroiodide or any other organic or inorganic salt.

For example to prepare hydrochloride salt, to a solution of compound (II) in solvent is added aqueous HCl or IPA-HCl over 30 to 60 minutes at ambient temperature 25-35° C. and further cooled at 10-20° C. and stirred for 2-4 hours. Organic solvent used for salt formation reaction is selected from group such as polar protic solvent alcohol, chlorinated hydrocarbons, aromatic or aliphatic hydrocarbon, ether, ester, nitrile, ketone, polar aprotic solvent or mixture thereof. Preferred solvent is ethyl acetate, cyclohexane. After completion of the reaction the hydrochloride salt is isolated by filtration. The solid obtained is washed with solvent, suck dried and dried in air oven to give hydrochloride salt of compound (II)

In first embodiment, the process of the present invention is as shown in Scheme VII and VIII.

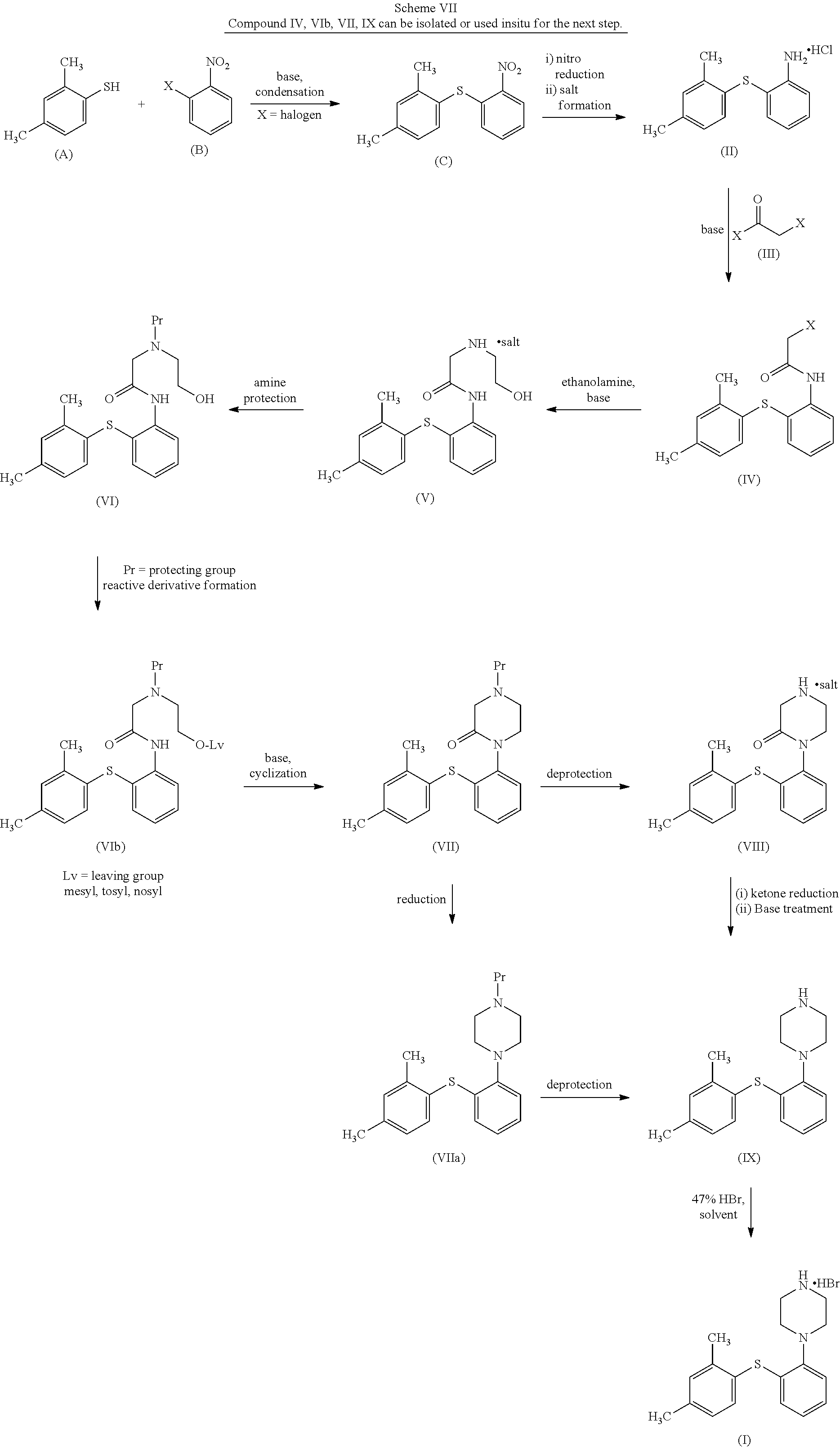

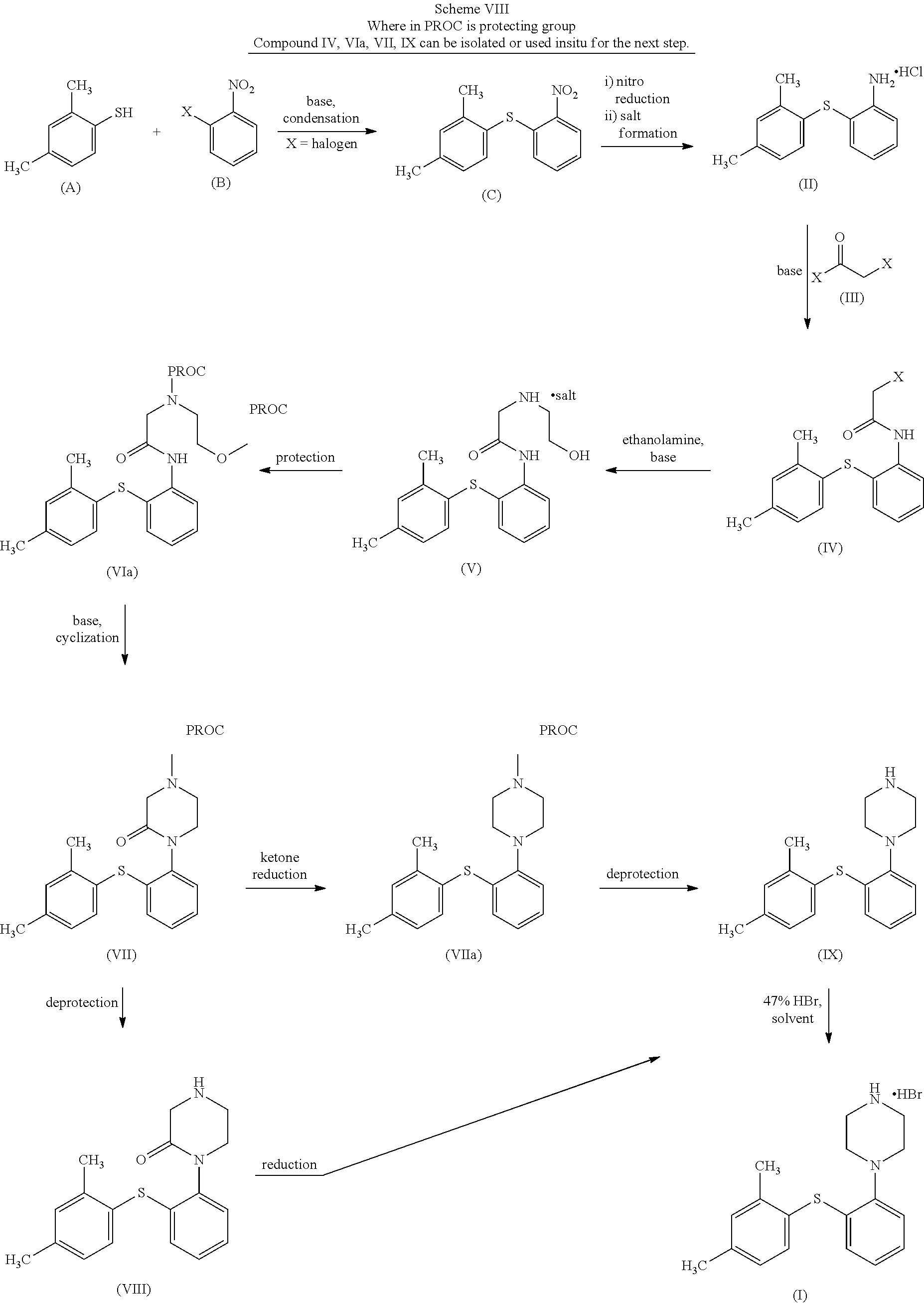

In second embodiment, the present invention provides a process for preparation of vortioxetine hydrobromide (I)

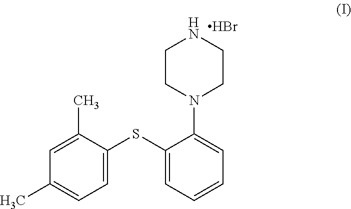 [SMILES: Br ; Cc1ccc(Sc2ccccc2N2CCNCC2)c(C)c1]

which comprises,
a) reacting 2-(2,4-dimethyl-phenylsulfanyl)-phenylamine (II) or its salt

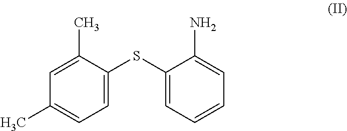 [SMILES: Cc1ccc(Sc2ccccc2N)c(C)c1]

with haloacetyl halide (XII)

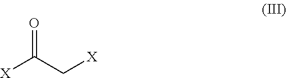

to give 2-halo-N-[2-(2,4-dimethyl-phenylsulfanyl)-phenyl]-acetamide (IV),

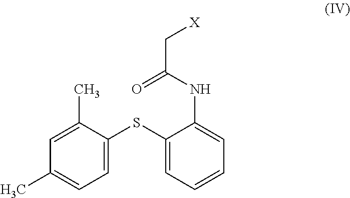

wherein X is halogen
b) reacting 2-halo-N-[2-(2,4-dimethyl-phenylsulfanyl)-phenyl]-acetamide (IV) with ethanolamine or its salt to give N-[2-(2,4-Dimethyl-phenylsulfanyl)-phenyl]-2-(2-hydroxy-ethylamino)-acetamide (V) or its salt,

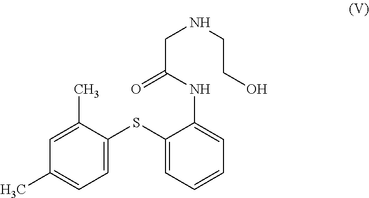 [SMILES: Cc1ccc(Sc2ccccc2NC(=O)CNCCO)c(C)c1]

c) cyclizing compound of formula (V) to give 1-[2-(2,4-dimethyl-phenylsulfanyl)-phenyl]-piperazin-2-one (VIII) or its salt,

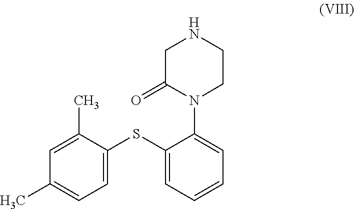 [SMILES: Cc1ccc(Sc2ccccc2N2CCNCC2=O)c(C)c1]

d) reducing compound of formula (VIII) to give vortioxetine (IX) or its salt, and

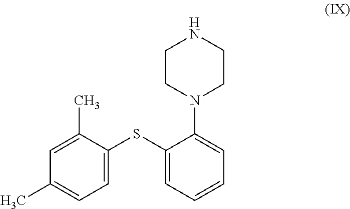 [SMILES: Cc1ccc(Sc2ccccc2N2CCNCC2)c(C)c1]

optionally converting vortioxetine (IX) or its salt to vortioxetine hydrobromide (I) Step a) and b) and d) can be performed according to the process as mentioned in one embodiment.

In step c), the cyclization of N-[2-(2,4-dimethyl-phenylsulfanyl)-phenyl]-2-(2-hydroxy-ethylamino)-acetamide (V) can be carried out using coupling reagent used for mitsunobu reaction in combination with phosphine reagent and organic solvent to give 1-[2-(2,4-dimethyl-phenylsulfanyl)-phenyl]-piperazin-2-one (VIII). Coupling reagent can be selected from diisopropylazodicarboxylate (DIAD), diethyl azodicarboxylate (DEAD), di-tert-butylazodicarboxylate, di-2-methoxyethyl azodicarboxylate (DMEAD), N,N,N′,N′-tetraisopropylazodicarboxamide (TIPA), N,N,N′,N′-Tetramethyl azodicarboxamide (TMAD), azopyridine, Di-(4-chlorobenzyl)azodicarboxylate (DCAD), 1,1′-(Azodicarbonyl)-dipiperidine (ADDP) and the like which are commonly used for mitsunobu reaction. Phosphine reagent used are selected from tri-n-butyl phosphine or triphenylphosphine (TPP), polymer-supported triphenylphosphine (PS-PPh₃), tributylphosphine (TBP) and the like. Organic solvent used is selected from chlorinated hydrocarbons such as dichloromethane, dichloroethane, chloroform or carbon tetrachloride; aromatic hydrocarbon such as toluene, xylene; ether such as dioxan, tetrahydrofuran (THF), methyl tertbutyl ether (MTBE), diethyl ether; nitrile such as acetonitrile; ester such as ethylacetate, isopropyl acetate; and the like; or mixtures thereof. The reaction is carried out at 0° C. to reflux for about 1-4 h. After completion of the reaction, conc HCl is added to obtain HCl salt of compound of formula (VIII)

In third embodiment, the present invention provides a process for preparation of vortioxetine hydrobromide salt (I)

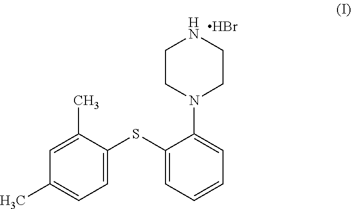 [SMILES: Br ; Cc1ccc(Sc2ccccc2N2CCNCC2)c(C)c1]

which comprises,
a) reacting 2-(2,4-dimethyl-phenylsulfanyl)-phenylamine (II) or its salt

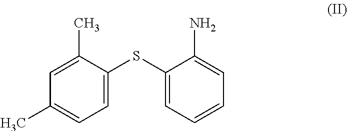 [SMILES: Cc1ccc(Sc2ccccc2N)c(C)c1]

with 2-halo-N-(2-hydroxyethy)-acetamide (X)

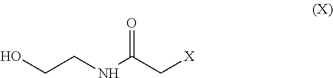

wherein X is halogen
to give 2-[2-(2,4-Dimethyl-phenylsulfanyl)-phenylamino]-N-(2-hydroxy-ethyl)-acetamide (XI) or its salt;

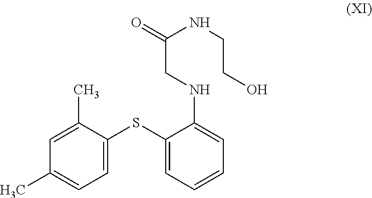 [SMILES: Cc1ccc(Sc2ccccc2NCC(=O)NCCO)c(C)c1]

b) cyclizing compound of formula (XI) to give 4-[2-(2,4-dimethyl-phenylsulfanyl)-phenyl]-piperazin-2-one (XII) or its salt,

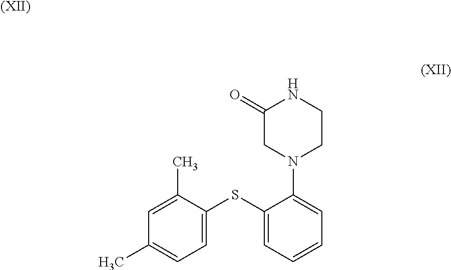

c) reducing compound of formula (XII) to give vortioxetine (IX), and

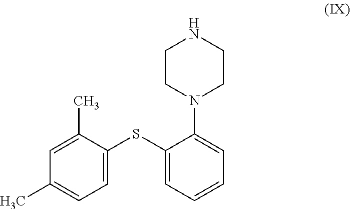 [SMILES: Cc1ccc(Sc2ccccc2N2CCNCC2)c(C)c1]

optionally converting it to vortioxetine hydrobromide (I)

In step a) 2-(2,4-dimethyl-phenylsulfanyl)-phenylamine (II) or its salt is reacted in presence of base and organic solvent with 2-halo-N-(2-hydroxyethy)-acetamide (X) to give 2-[2-(2,4-Dimethyl-phenylsulfanyl)-phenylamino]-N-(2-hydroxy-ethyl)-acetamide (XI). Base may be selected from any organic base or inorganic base as mentioned at the beginning of the detail description. Preferred base is hydroxide, carbonate, bicarbonate or hydride of alkali or alkaline earth metal. It is used in the form of an aq. solution. For example, aq. Sodium or potassium carbonate, aq. Sodium or potassium bicarbonate may be used. Preferred solvent are chlorinated hydrocarbons such as dichloromethane, dichloroethane, chloroform and carbon tetrachloride. Other solvent such as aromatic hydrocarbon, ether, nitrile, ester, ketone may also be used. The reaction is carried out from ambient temperature to reflux for about 6 to 8 h. After completion of the reaction, the mixture is washed with water then conc. HCl is added to give HCl salt of compound of formula (XI).

In step b), Cyclization may be carried out directly using coupling agent or via preparation of reactive derivative of compound of formula (XI) by following Scheme IX.

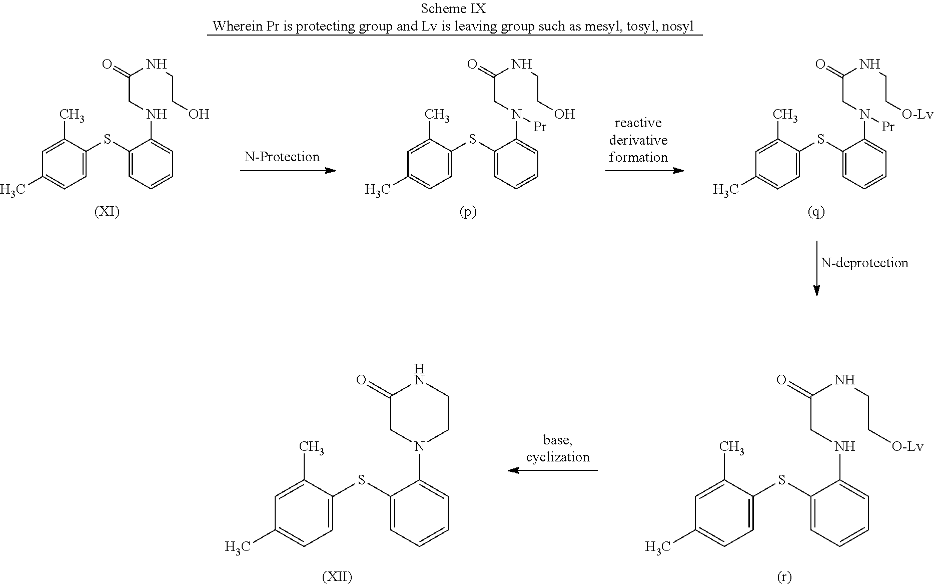

2-[2-(2,4-Dimethyl-phenylsulfanyl)-phenylamino]-N-(2-hydroxy-ethyl)-acetamide (XI) is reacted with protecting reagent having protecting group (Pr) to give compound of formula (p). Protecting group can be tertbutyloxycarbonyl (boc), triphenylmethyl (trityl), benzyloxycarbonyl (cbz), benzyl, trifluoroacetyl (COCF₃), acetyl, silyl and the like. Appropriate protecting reagent is used to induce specific protecting group. For example boc anhydride is used for boc protection, trityl chloride or benzyl chloride is used for trityl or benzyl protection respectively, trifluoroacetyl chloride or acetyl chloride used for trifluoroacetyl (COCF₃) or acetyl protection respectively. The reaction is carried out in an organic solvent in presence of a base. Preferred base is organic base such as triethylamine (TEA), diethylamine (DEA), pyridine, 1,8-diazabicyclo[5.4.0]undec-7-ene (DBU) and 1,4-diazabicyclo[2.2.2]octane (DABCO) and the like or mixtures thereof. The preferred organic solvent used for this step may be selected from chlorinated hydrocarbons such as dichloromethane, dichloroethane, chloroform or carbon tetrachloride.

The cyclization of compound of formula (XI) can be carried out by first converting compound of formula (p) into a reactive derivative of hydroxyl group of compound (p) followed by deprotecting the protecting group Pr of compound (q) to give compound (r), which in turn is cyclized to give compound of formula (XII) or its salt as shown in above scheme. This is done by converting hydroxyl group of compound (XI) to O-mesyl, O-tosyl or O-nosyl group which is suitable leaving group.

Compound of formula (XI) is converted to its reactive derivative compound of formula (r) by doing sulfonylation at hydroxyl group of compound (p) in presence of a base in an organic solvent. This is done by converting hydroxyl group of compound (XI) to O-mesyl, O-tosyl or O-nosyl group which is suitable leaving group suitable leaving group suitable leaving group. Various sulfonylating reagents can be used such as methane sulfonyl chloride (mesyl chloride), p-toluene sulfonyl chloride (tosyl chloride) or nosyl chloride to prepare reactive derivative of formula (q) wherein leaving group (Lv) is mesyl, tosyl or nosyl obtained according to the sulfonylating reagent used. sulfonylating reagent is used in 1.5 to 2.5 mol equivalent to compound (p). Preferred base is organic base such as triethylamine (TEA), diethylamine (DEA), pyridine, 1,8-diazabicyclo[5.4.0]undec-7-ene (DBU) and 1,4-diazabicyclo[2.2.2]octane (DABCO) and the like or mixtures thereof. Preferred organic solvent is chlorinated hydrocarbons such as dichloromethane, dichloroethane, chloroform or carbon tetrachloride or mixture thereof.

Further deprotection of compound (q) by removing protecting group Pr in an organic solvent gives a reactive derivative (r), for example Methanesulfonic acid 2-{2-[2-(2,4-dimethyl-phenylsulfanyl)-phenylamino]-acetylamino}-ethyl ester is obtained by deprotection of (q). Deprotection can be affected via acid or base or hydrogenolysis, depending upon the protecting group Pr. If protecting group Pr is boc or trityl, then it will be deprotected by an acid. If protecting group Pr is benzyl or cbz, then it can be removed by hydrogenation. Protection, deprotection reaction is well described in the literature. Preferred solvent is selected from ester such as ethyl acetate, isopropyl acetate. Other solvent from group such as polar protic solvent alcohol, chlorinated hydrocarbons, aromatic hydrocarbon, ether, nitrile, ketone, polar aprotic solvent, water or mixture of solvent with water can also be used. Acid used may be organic or mineral acid. It can be aqueous or concentrated solution such as conc. HCl or saturated in solvent such as isopropanol-HCl. Compound (q) where protecting group (Pr) is boc can be deprotected by isopropanol-HCl in a solvent. Subsequently compound (r) is cyclized to 4-[2-(2,4-Dimethyl-phenylsulfanyl)-phenyl]-piperazin-2-one (XII) or its salt by heating the solution of compound (r) in organic solvent with base. Preferred base are inorganic base from group alkoxide, hydroxide, carbonate, bicarbonate or hydride of alkali or alkaline earth metal. Preferred base is selected from potassium carbonate, sodium carbonate, sodium bicarbonate, potassium bicarbonate, sodium hydroxide or potassium hydroxide. Preferred organic solvent used for cyclization is polar aprotic solvent such as dimethyl formamide (DMF), dimethyl sulfoxide (DMSO), dimethyl acetamide (DMAc), N-methyl pyrrolidine (NMP) or mixtures thereof.

In other alternative, the direct cyclization of compound of formula (XI) can be carried out using coupling reagent used for mitsunobu reaction in combination with phosphine reagent and organic solvent to give compound of formula (XII). Coupling reagent can be selected from diisopropylazodicarboxylate (DIAD), diethyl azodicarboxylate (DEAD), di-tert-butylazodicarboxylate, di-2-methoxyethyl azodicarboxylate (DMEAD), N,N,N′,N′-tetraisopropylazodicarboxamide (TIPA), N,N,N′,N′-Tetramethyl azodicarboxamide (TMAD), azopyridine, Di-(4-chlorobenzyl)azodicarboxylate (DCAD), 1,1′-(Azodicarbonyl)-dipiperidine (ADDP) and the like which are commonly used for mitsunobu reaction. Phosphine reagent used are selected from tri-n-butyl phosphine or triphenylphosphine (TPP), polymer-supported triphenylphosphine (PS-PPh₃), tributylphosphine (TBP) and the like. Organic solvent used is selected from chlorinated hydrocarbons such as dichloromethane, dichloroethane, chloroform or carbon tetrachloride; aromatic hydrocarbon such as toluene, xylene; ether such as dioxan, tetrahydrofuran (THF), methyl tertbutyl ether (MTBE), diethyl ether; nitrile such as acetonitrile; ester such as ethylacetate, isopropyl acetate; and the like; or mixtures thereof. The reaction is carried out at 0° C. to reflux for about 1-4 h. After completion of the reaction, conc HCl is added to obtain HCl salt of compound of formula (XII)

In step c), the reduction of 4-[2-(2,4-Dimethyl-phenylsulfanyl)-phenyl]-piperazin-2-one (XII) gives vortioxetine (IX). The crude product obtained thereafter may optionally be used for salt formation with or without further purification. Reduction is carried out in the same manner as described before in first embodiment for reduction of compound of formula (IX) using reducing reagents and solvent as mentioned in that embodiment.

Vortioxetine (IX) may be optionally converted to vortioxetine hydrobromide (I) by process known in the art.

Starting compound 2-halo-N-(2-hydroxyethy)-acetamide (X) can be prepared by reacting ethanolamine with haloacetyl halide (III) in organic solvent with base as shown below scheme 3b. Organic solvent used may be selected from chlorinated hydrocarbons such as dichloromethane, dichloroethane, chloroform and carbon tetrachloride. Other solvent such as aromatic hydrocarbon, ether, nitrile, ester, ketone can also be used. Base can be selected from such as diisopropylethylamine (DIPEA), triethylamine (TEA), diethylamine (DEA), pyridine

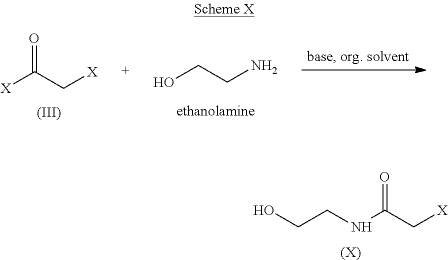

In third embodiment, the process of the present invention is as shown in Scheme-XI.

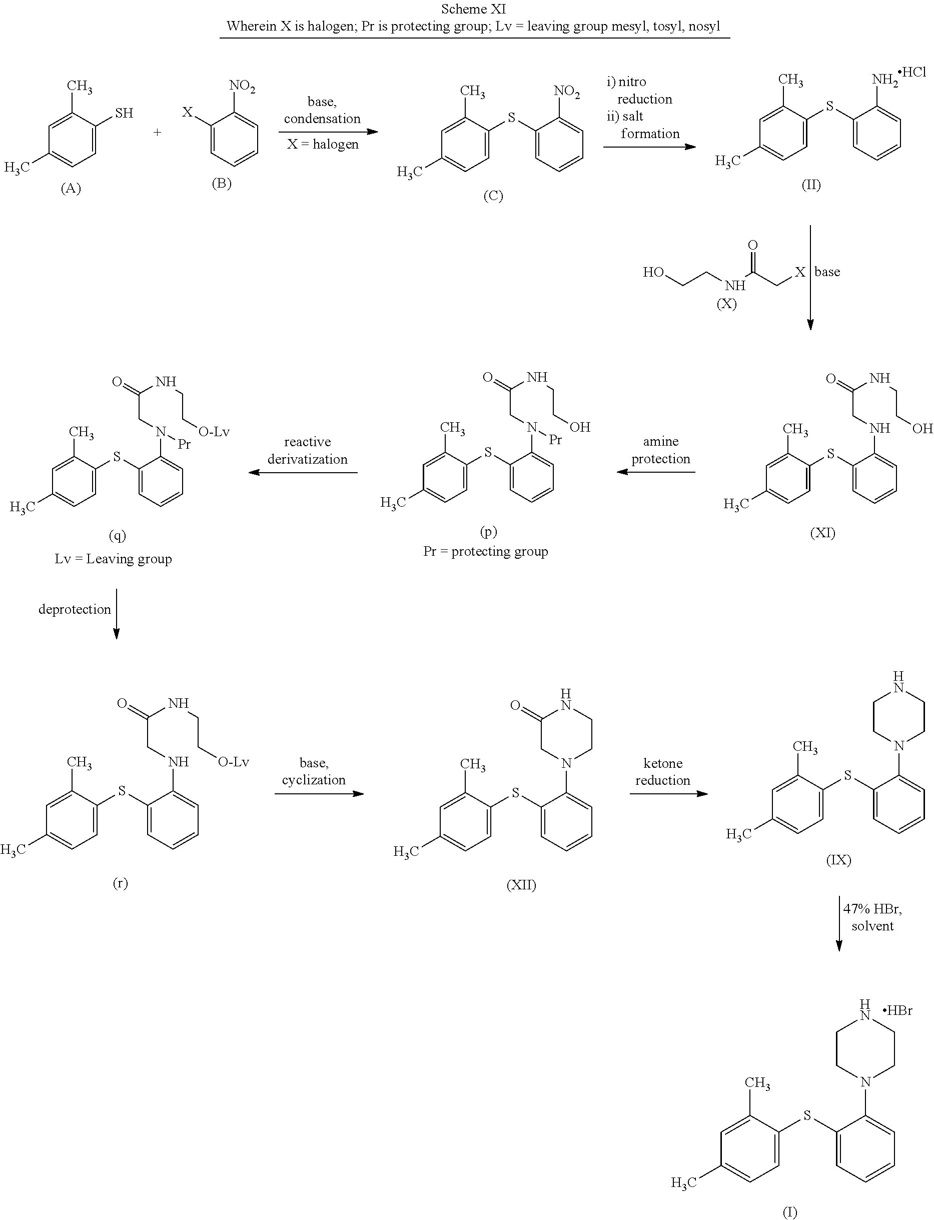

In fourth embodiment, the present invention provides a process for preparation of vortioxetine hydrobromide (I)

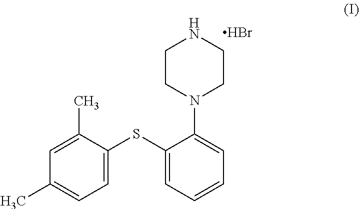 [SMILES: Br ; Cc1ccc(Sc2ccccc2N2CCNCC2)c(C)c1]

which comprises,
a) reacting 1-halo-2,4-dimethylbenzene (XIII)

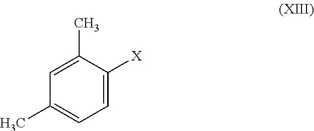

with 2-halobenzenethiol (XIV)

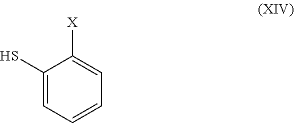

to give 1-(2-halo-phenylsulfanyl)-2,4-dimethyl-benzene (XV);

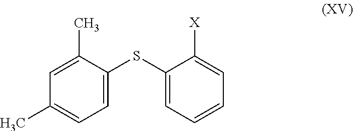

wherein X is halogen
b) reacting 1-(2-halo-phenylsulfanyl)-2,4-dimethyl-benzene (XV) with piperazine 2-one (XVI)

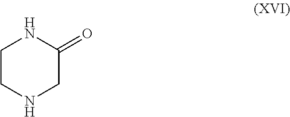

to give 4-[2-(2,4-Dimethyl-phenylsulfanyl)-phenyl]-piperazin-2-one (XII);

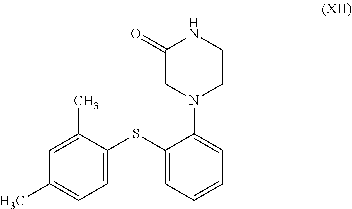 [SMILES: Cc1ccc(Sc2ccccc2N2CCNC(=O)C2)c(C)c1]

c) reducing 4-[2-(2,4-Dimethyl-phenylsulfanyl)-phenyl]-piperazin-2-one (XII) to vortioxetine (IX) and

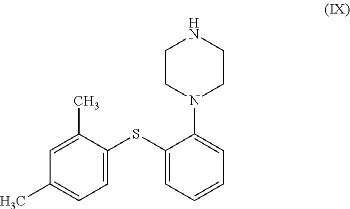 [SMILES: Cc1ccc(Sc2ccccc2N2CCNCC2)c(C)c1]

d) optionally converting vortioxetine (IX) to vortioxetine hydrobromide (I).

In step a) 1-halo-2,4-dimethylbenzene (XIII) is reacted with 2-halobenzenethiol (XIV) to give 1-(2-halo-phenylsulfanyl)-2,4-dimethyl-benzene (XV). The halogen is selected from Cl, Br, I, F. Halogen atom in compound (XIII) and (XIV) can be same or different. The compound (XV) can be obtained according to the halogen taken in compound (XIV). The reaction is carried out using palladium catalyst, base and organic solvent. The palladium catalyst consists of a palladium source and a phosphine ligand. Useful palladium sources include palladium in different oxidations states, such as e.g. 0 and II. Examples of palladium sources which may be used in the process of the present invention are Pd(dba)₂, Pd₂(dba)₃, Pd(OAc)₂, Pd(dppf)Cl₂. The “dba” abbreviates dibenzylideneacetone. “Ac” abbreviates acetyl. The palladium source is typically applied in an amount of about 0.1 mol % to about 15 mol %, preferably in an amount of about 0.1 mol % to about 10 mol %. Hereinafter the mol % as mentioned in specification is calculated with respect to the limiting reactant.

Numerous phosphine ligands are known, both monedentate and bidentate. Useful phosphine ligands include racemic 2,2′-bis-diphenylphosphanyl-[1,1′]binaphtalenyl(rac-BINAP), 1,1′-bis(diphenylphosphino)ferrocene (DPPF), bis-(2-diphenylphosphinophenyl)ether (DPEphos), triphenyl phosphine (TPP), tri-t-butyl phosphine (Fu's salt), biphenyl-2-yl-di-t-butyl-phosphine, biphenyl-2-yl-dicyclohexyl-phosphine, (2′-dicyclohexylphosphanyl-biphenyl-2-yl)-dimethyl-amine, [2′-(di-t-butyl-phosphanyl)-biphenyl-2-yl]-dimethyl-amine, and dicyclohexyl-(2′,4′,6′-tri-propyl-biphenyl-2-yl)-phosphane Moreover, carbene ligands, such as e.g. 1,3-bis-(2,6-di-isopropyl-phenyl)-3H-imidazol-1-ium chloride may be used instead of phosphine ligands. In one embodiment, the phosphine ligand is rac-BINAP, DPPF or DPEphos, and in particular rac-BINAP. The phosphine ligand is usually applied in an amount of about 0.1 mol % to about 10 mol %, preferably, about 1 mol % to about 5 mol %, more preferably, about 1-2 mol %

The solvent used for this step may be selected from aprotic organic solvents, polar aprotic or polar protic solvent or mixtures thereof. Typically, the solvent is selected from amongst toluene, xylene, triethyl amine, tributyl amine, dioxan, N-methylpyrrolidone, N,N-dimethylformamide (DMF), dimethyl sulfoxide (DMSO), dimethyl acetamide (DMAc), alcohol, acetonitrile, THF or from any mixture thereof. Particular mentioning is made of toluene as solvent.

The base used is selected from inorganic or organic base. Inorganic base are alkoxide, hydroxide, carbonate, bicarbonate or hydride of alkali or alkaline earth metal. Inorganic base are selected from sodium tert butoxide, potassium tert butoxide, sodium methoxide, sodium ethoxide, potassium methoxide, potassium ethoxide, sodium hydroxide, potassium hydroxide, lithium hydroxide, cesium carbonate, potassium carbonate, sodium carbonate, sodium bicarbonate, potassium bicarbonate, sodium hydride, potassium hydride, lithium hydride and the like or mixtures thereof. Organic base are selected from triethylamine (TEA), diethylamine (DEA), pyridine, 1,8-diazabicyclo[5.4.0]undec-7-ene (DBU) and 1,4-diazabicyclo[2.2.2]octane (DABCO) and the like or mixtures thereof. Typically, the base is added is an amount around 1-5 equivalents, such as 1-3 equivalents, such as 2-3 equivalents.

The preferable reagent combination is Pd(dba)₂, rac-BINAP and sodium tert butoxide and solvent is toluene.

The reaction is carried out in an inert gas atmosphere such as nitrogen gas, argon gas. The reaction is carried out at an elevated temperature condition at about 70° C. to about 120° C., preferably from about 90° to about 100° C. Customary work up like quenching with water, extraction in solvent and evaporation of the solvent provides compound of formula (XV) which can be used for next step without further purification.

In step b) 1-(2-halo-phenylsulfanyl)-2,4-dimethyl-benzene (XV) is reacted with piperazine 2-one (XVI) to give 4-[2-(2,4-Dimethyl-phenylsulfanyl)-phenyl]-piperazin-2-one (XII). The reaction is carried out using palladium catalyst, base and organic solvent. The palladium catalyst consists of a palladium source and a phosphine ligand. The examples of palladium source, phosphine ligands, base and solvent are as given above for step a). The reaction is carried out under inert atmosphere. The reaction is carried out at temperature condition from 70° C. to 120° C., preferably from 90° to 100° C.

Step c) reduction of 4-[2-(2,4-Dimethyl-phenylsulfanyl)-phenyl]-piperazin-2-one (XII) to vortioxetine (IX) is described in third embodiment.

In step (d), vortioxetine base obtained in step (c) is converted to vortioxetine hydrobromide by process known in the art.

In fourth embodiment, the process of the present invention is as shown in Scheme XII.

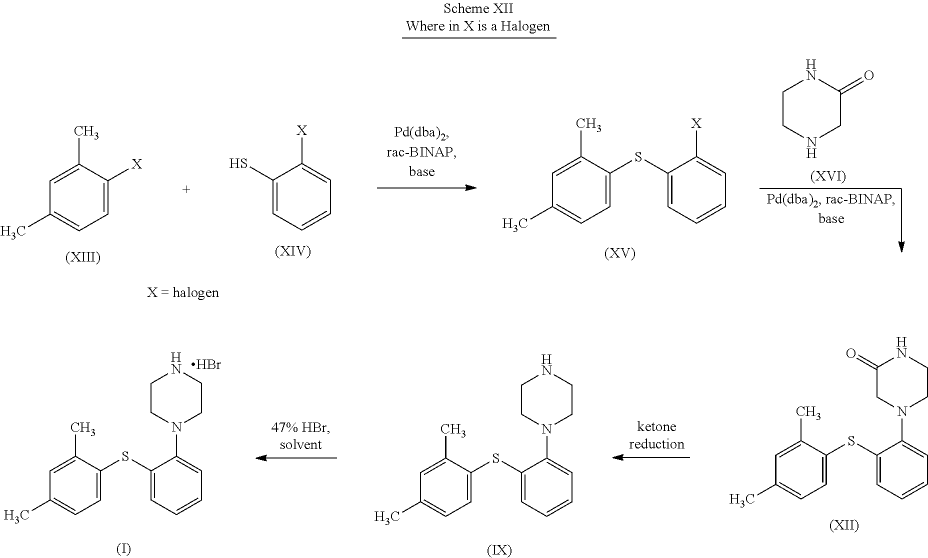

The following examples are given for the purpose of illustrating the present invention and should not be considered as limitation on the scope or spirit of the invention.

### Example 1

### Preparation of 2,4-Dimethyl-1-(2-nitro-phenylsulfanyl)-benzene (C)

To a stirred solution of 2,4-dimethylbenzenethiol (100 g, 0.723 mol) in methanol (500 ml) was added aqueous NaOH solution (1.085 mol in 200 ml water) at 25-35° C. The reaction mixture was heated at 60-70° C.1-Chloro-2-nitro-benzene (125.4 g, 0.795 mol) in solution of methanol was added to the reaction mixture and at 60-70° C. and further reaction mass was stirred for 4 to 12 h at at 60-70° C. Completion of the reaction was confirmed using thin layer chromatography (TLC). After completion of the reaction, the reaction mixture was cooled at 25-35° C. and filtered. The solid obtained was washed with methanol (50 ml), suck dried. The above wet cake was charged in to methanol (300 ml) at 25-35° C. and further stirred for 30 minutes. The product was filtered and washed with methanol (2×50 ml), suck dried. Further wet cake was charged in to water (500 ml) at 25-35° C. and reaction mass was stirred for 30 minutes. The product was filtered and washed with water (2×50 ml), suck dried, then dried in oven to give the title product as yellowish crystalline solid (165.4 g). The compound was used for next step without further purification.

Yield: 88.0%

### Example 2

### Preparation of 2-(2,4-Dimethyl-phenylsulfanyl-phenylamine hydrochloride (II)

To a stirred solution of 2,4-Dimethyl-1-(2-nitro-phenylsulfanyl)-benzene (100 g, 0.386 mol) in methanol (500 ml) was added zinc dust (88.24 g, 1.349 mole) at 25-35° C. and stirred. The reaction mixture was heated at 60-70° C. Aq ammonium chloride solution (51.6 g, 0.964 mole, 150 ml) was added to the reaction mixture and heated at 60-70° C. for 6-10 h. Completion of the reaction was confirmed using thin layer chromatography (TLC). After completion of the reaction, methanol (300 ml) was charged and the reaction mixture was filtered through hyflo bed to remove salt. The hyflo bed was washed with methanol (100 ml×2). Combined filtrate was evaporated up to thick residue, further ethylacetate (100 ml) was charged and evaporated up to thick residue. The residue was charged with ethylacetate (300 ml) and filtered to remove undissolved solid. The filtrate was washed with water (300 ml×2). Organic phase was separated and cyclohexane (750 ml) was added to the organic phase at ambient temperature 25° C. to 35° C. Aqueous hydrochloric acid (0.617 mole) was added over 30 min to the reaction mixture and further the reaction mass was cooled to 10° C. to 20° C. the stirred at 15° C. to 25° C. for 2-4 h. The reaction mass was filtered and solid obtained was washed with mixture of ethyl acetate and cyclohexane (3×100 ml), suck dried and dried in air oven to give the title product as white solid (86.7 g)

Yield: 85%

### Example 3

### Preparation of 2-Chloro-N-[2-(2,4-dimethyl-phenylsulfanyl)-phenyl]-acetamide (IV)

To a stirred solution of 2-(2,4-Dimethyl-phenylsulfanyl)-phenylamine hydrochloride (100 g, 0.376 mole) in dichloromethane (1000 ml) was added aqueous KHCO₃ solution (100.0 g, 1.000 mol, in 500 ml water) over 10-20 minutes at 25° C. to 35° C. The reaction mixture was cooled at 0° C. to 10° C. Chloroacetyl chloride (57.30 g, 0.507 mole) was added to the reaction mixture and stirred at 0-10° C. for 1 to 2 h. Completion of reaction was confirmed using thin layer chromatography (TLC). After completion of the reaction, water (600 ml) was added to reaction mixture and extracted. The organic phase was separated, dried over sodium sulfate and evaporated to give the title product as off white solid (97.75 g).

Yield: 85%

### Example 4

### Preparation of N-[2-(2,4-Dimethyl-phenylsulfanyl)-phenyl]-2-(2-hydroxy-ethylamino)-acetamide hydrochloride (V)

To a stirred solution of 2-Chloro-N-[2-(2,4-dimethyl-phenylsulfanyl)-phenyl]-acetamide (100 g, 0.326 mol) in isopropyl acetate (800 ml) was added ethanol amine (91.62 g, 1.499 mol) at 25° C. to 35° C. The reaction mixture was heated at 70-80° C. for 6 to 8 h. Completion of reaction was confirmed using thin layer chromatography (TLC). After completion of the reaction, the reaction mixture was cooled at 25° C. to 35° C. and quenched with 1 N HCl (1000 ml). Aq. phase was separated and water (500 ml) was added to it and stirred at 25° C. to 35° C. for 1 h. The reaction mixture was filtered, suck dried and dried in oven to give the title product as off white solid (102.0 g).

Yield: 85.0%

### Example 5

### Preparation of N-[2-(2,4-Dimethyl-phenylsulfanyl)-phenyl]-2-(2-hydroxy-ethylamino)-acetamide hydrochloride (V)

To a stirred solution of 2-(2,4-Dimethyl-phenylsulfanyl)-phenylamine hydrochloride (100 g, 0.376 mole) in toluene (950 ml) was added aqueous KHCO₃ solution (120.0 g, 1.203 mol, in 600 ml water) over 10-20 minutes at 25° C. to 35° C. The reaction mixture was heated to 30° C. to 40° C. and stirred for 60 minutes at 30° C. to 40° C. The reaction mixture was cooled at 0° C. to 10° C. Solution of chloroacetyl chloride (57.30 g, 0.507 mole) in toluene (50 ml) was added to the reaction mixture and stirred at 0-10° C. for 1 to 2 h. Completion of reaction was confirmed using thin layer chromatography (TLC). After completion of the reaction, reaction mass temperature was raised to 25-35° C. and stirred. The organic phase was separated. To organic layer containing of 2-Chloro-N-[2-(2,4-dimethyl-phenylsulfanyl)-phenyl]-acetamide(IV) in toluene was added ethanol amine (105.7 g, 1.730 mol) at 25° C. to 35° C. The reaction mixture was heated at 100-110° C. for 6 to 8 h. Completion of reaction was confirmed using thin layer chromatography (TLC). After completion of the reaction, the reaction mixture was cooled at 25° C. to 35° C. and washed with water (2×500 ml). The organic phase was separated and the temperature was raised at 40° C. to 50° C. Aqueous hydrochloric acid (63.0 g, 0.602 mole) was added within 30 minutes at 40° C. to 50° C. and the reaction mass was stirred for 2-4 hr at 40° C. to 50° C. The reaction mixture was filtered, suck dried and product was washed with toluene (2×100 ml), dried in oven to give the title product as off white solid (102.0 g).

Yield: 85.0%

### Example 6

### Preparation of {[2-(2,4-Dimethyl-phenylsulfanyl)-phenylcarbamoyl]-methyl}-(2-hydroxy-ethyl)-carbamic acid tert-butyl ester [Compound (VI) Wherein Pr is Boc]

To a stirred mixture of N-[2-(2,4-Dimethyl-phenylsulfanyl)-phenyl]-2-(2-hydroxy-ethylamino)-acetamide hydrochloride (100 g, 0.272 mol) and toluene (1000 ml) was added triethylamine (69.0 g, 0.681 mol) at 25° C. to 35° C. and stirred for 30 minutes. Boc-anhydride (83.3.50 g, 0.381 mol) was added at 25° C. to 35° C. The reaction mixture was stirred at 25° C. to 35° C. for 4 to 8 h. Completion of reaction was confirmed using thin layer chromatography (TLC). After completion of the reaction, the reaction mass washed with aqueous hydrochloric acid (500 ml) and further washed with water (500 ml) at 25° C. to 35° C. The organic phase was separated and evaporated. Toluene (50 ml) was added to the reaction mass at 40° C. to 50° C. and further stirred for 60 minutes. The reaction mixture was cooled to 25° C. to 35° C. and cyclohexane (500 ml) was charged. The reaction mass was stirred for 2-4 hr at 25° C. to 35° C. The reaction mass was filtered and suck dried, washed the product with cyclohexane (2×100 ml), dried in oven to give the title product as off white solid (102.0 g).

Yield: 87%

### Example 7

### Preparation of Methanesulfonic acid 2-(tert-butoxycarbonyl-{[2-(2,4-dimethyl-phenylsulfanyl)-phenylcarbamoyl]-methyl}-amino)-ethyl Ester (VIb)

To a stirred mixture of {[2-(2,4-Dimethyl-phenylsulfanyl)-phenylcarbamoyl]-methyl}-(2-hydroxy-ethyl)-carbamic acid tert-butyl ester (100 g, 0.232 mol), triethylamine (46.95 g, 0.464 mol) and toluene (1000 ml) was added methane sulphonyl chloride (37.20 g, 0.324 mol) at 0-10° C. and stirred for 2-3 hours at the same temperature. Completion of the reaction was confirmed using thin layer chromatography (TLC). After completion of the reaction, water (1000 ml) was added to the reaction mixture and extracted. Organic phase was separated, dried over sodium sulfate and evaporated to give the title product as oily mass (110.0 g).

Yield: 93%

### Example 8

### Preparation of 4-[2-(2,4-Dimethyl-phenylsulfanyl)-phenyl]-3-oxo-piperazine-1-carboxylic acid tert-butyl Ester (VIII)

A mixture of methanesulfonic acid 2-(tert-butoxycarbonyl-{[2-(2,4-dimethyl-phenylsulfanyl)-phenylcarbamoyl]-methyl}-amino)-ethyl ester (100 g, 0.196 mol) in toluene (500 ml) and potassium tertiary butoxide (52.19 g, 0.465 mole) was stirred at 25° C. to 35° C. for 2-3 h. Completion of the reaction was confirmed using thin layer chromatography (TLC). After completion of reaction, the reaction mixture was quenched with water (1000 ml) and extracted. The organic phase was separated, dried on sodium sulfate and evaporated to give the title compound as off white solid (60.75 g). The crude compound was used for next step without further purification

Yield: 75.0%

### Example 9

### Preparation of 4-[2-(2,4-Dimethyl-phenylsulfanyl)-phenyl]-3-oxo-piperazine-1-carboxylic acid tert-butyl Ester [Compound (VII) Wherein Pr is Boc]

To a stirred mixture of {[2-(2,4-Dimethyl-phenylsulfanyl)-phenylcarbamoyl]-methyl}-(2-hydroxy-ethyl)-carbamic acid tert-butyl ester (100 g, 0.232 mol) and toluene (950 ml) was added triethylamine (47.0 g, 0.464 mol) at 0° C. to 10° C. Solution of methane sulphonyl chloride (37.30 g, 0.325 mol) in toluene (50 ml) was added at 0° C. to 10° C. and stirred for 2-3 hours at the same temperature. Completion of the reaction was confirmed using thin layer chromatography (TLC). After completion of the reaction, solution of sodium chloride (100 g) in water (500 ml) was added to the reaction mixture and extracted. Organic phase was separated, and cooled to 15° C. to 25° C., potassium tertiary butoxide (39.1 g, 0.348 mole) was added at 15° C. to 25° C. The reaction mass temperature was raised to 25° C. to 35° C. and stirred the reaction mass for 4-8 h at same temperature. Completion of the reaction was confirmed using thin layer chromatography (TLC). After completion of reaction, the reaction mixture was quenched with water (750 ml) and extracted. The organic phase was separated, and washed with water (750 ml). The organic phase was separated and evaporated. Cyclohexane (100 ml) was added to the reaction mass and evaporated, further cyclohexane (300 ml) was added to the residue at 25° C. to 35° C. and stirred for 2-3 h at same temperature. The reaction mass was filtered, suck dried, washed with cyclohexane (2×50 ml), dried in oven to give the title compound as off white solid (74.0 g).

Yield: 77.0%

### Example 10

### Preparation of 1-[2-(2,4-Dimethyl-phenylsulfanyl)-phenyl]-piperazin-2-one hydrochloride (VIII)

To a stirred solution of 4-[2-(2,4-Dimethyl-phenylsulfanyl)-phenyl]-3-oxo-piperazine-1-carboxylic acid tert-butyl ester (100 g, 0.242 mole) in ethyl acetate (400 ml) was heated to 40° C. to 50° C. Aqueous hydrochloric acid (55.7 g, 0.533 mole) was added over 30 minutes at 40° C. to 50° C. and stirred further for 2 to 4 h. Completion of the reaction was confirmed using thin layer chromatography (TLC). After completion of the reaction, the reaction mixture was cooled to 25° C. to 35° C. and further stirred for 2-3 h, filtered, obtained solid was washed with ethylacetate (2×100 ml), suck dried and dried in oven at 40° C. to 45° C. to give the title product (65.0 g)

Yield: 76%

### Example 11

### Preparation of 1-[2-(2,4-Dimethyl-phenylsulfanyl)-phenyl]-piperazine i.e. vortioxetine (IX)

A solution of 1-[2-(2,4-Dimethyl-phenylsulfanyl)-phenyl]-piperazin-2-one hydrochloride (100 g, 0.286 mol) in Tetrahydrofuran (500 ml) was cooled at 0 to −10° C. 10M Borane-DMS solution (86.0 ml, 0.856 mol) was added to the reaction mixture and gradually heated at 50 to 60° C. for 3 to 4 h. Completion of the reaction was confirmed using thin layer chromatography (TLC). After completion of the reaction, the reaction mixture was cooled at 0° C. to 5° C. 10% aq. HCl solution (200 ml) was added to the reaction mixture and heated at 50-60° C. for 2 h. The reaction mixture was cooled at ambient temperature 25° C. to 35° C. and basified with 10% aq. NaOH solution till pH 10 to 12 was obtained. The reaction mixture was extracted in ethylacetate (500 ml). Organic phase was separated, washed with water (250 ml), dried over sodium sulfate and evaporated to give the title product (73.0 g).

Yield: 85.0%

### Example 12

### Preparation of Vortioxetine Hydrobromide (I)

To a stirred solution of 1-[2-(2,4-Dimethyl-phenylsulfanyl)-phenyl]-piperazine (100 g, 0.335 mol) in ethyl acetate (500 ml) was added 47% Hydrobromic acid (86.50 g, 0.502 mol) over 20-30 minutes at 25-35° C. The reaction mixture was stirred at 25-35° C. for 1 to 2 h. The reaction progress was monitored on thin layer chromatography (TLC). After completion of the reaction, the reaction mixture was filtered. The solid obtained was washed with ethyl acetate (100 ml), suck dried and dried in oven 40-50° C. for 6-8 h to give the title product (108.0 g).

Yield: 85%

### Example 13

### Preparation of 1-[2-(2,4-Dimethyl-phenylsulfanyl)-phenyl]-piperazine i.e. vortioxetine hydrobromide (I) Solvate

A solution of 1-[2-(2,4-Dimethyl-phenylsulfanyl)-phenyl]-piperazin-2-one hydrochloride (100 g, 0.286 mol) in MDC (1000 ml) was added 10M Borane-DMS solution (45.8 g, 0.573 mol) was added to the reaction mixture within 30 minutes. The reaction mixture was heated at 35 to 45° C. for 4 to 8 h. Completion of the reaction was confirmed using thin layer chromatography (TLC). After completion of the reaction, the reaction mixture was cooled at 0° C. to 10° C. Solution of aqueous hydrochloric acid solution (59.8 g) in water (250 ml) was added to the reaction mixture slowly and reaction mixture was heated at 35-45° C. for 2-3 h. The reaction mixture was cooled at ambient temperature 25° C. to 35° C. and basified with 20% aq. NaOH solution till pH 8.5 to 10.5 was obtained. The reaction mixture was stirred and organic phase was separated, washed with 20% NaCl solution (2×500 ml), the organic phase was separated and evaporated to give the residue. To the residue 2-butanol (1200 ml) was charged and reaction mass was heated to 60 to 70° C., solution was filtered and filtrate was heated to 60 to 70° C. 47% Hydrobromic acid (51.40 g, 0.298 mol) over 20-30 minutes at 60 to 70° C. The reaction mixture was stirred at 60 to 70° C. for 1 h and then cooled to 25 to 35° C., stirred at same temperature for 1-2 h. The reaction mixture was filtered, the solid obtained was washed with 2-butanol (2×100 ml), suck dried and dried in oven 40-50° C. for 6-10 h to give the title product (104.0 g).

Yield: 80%

### Example 14

### Preparation of Methanesulfonic acid 2-({[2-(2,4-dimethyl-phenylsulfanyl)-phenylcarbamoyl]-methyl}-methanesulfonyl amino)-ethyl Ester [Compound (VIa) Wherein Pr is methanesulfonyl]

To a stirred mixture of N-[2-(2,4-Dimethyl-phenylsulfanyl)-phenyl]-2-(2-hydroxy-ethylamino)-acetamide hydrochloride (100 g, 0.272 mol), triethylamine (165.0 g, 1.632 mol) and dichloromethane (1000 ml) was added methane sulphonyl chloride (93.5 g, 0.816 mol) at 0-5° C. and stirred for 1-2 hours at the same temperature. Completion of the reaction was confirmed using thin layer chromatography (TLC). After completion of the reaction, water (500 ml) was added to the reaction mixture and extracted. Organic phase was separated, washed with water (500 ml), dried over sodium sulfate and evaporated to give the title product as oily mass (120.1 g).

Yield: 90%

### Example 15

### Preparation of 1-[2-(2,4-Dimethyl-phenylsulfanyl)-phenyl]-4-methanesulfonyl piperazin-2-one [Compound (VII) Wherein Pr is methanesulfonyl]

A mixture of Methanesulfonic acid 2-({[2-(2,4-dimethyl-phenylsulfanyl)-phenylcarbamoyl]-methyl}-methanesulfonyl amino)-ethyl ester (100 g, 0.205 mol), dimethyl formamide (500 ml) and potassium carbonate (83.63 g, 0.606 mole) was heated at 50° C. to 60° C. for 2-3 h. Completion of the reaction was confirmed using thin layer chromatography (TLC). After completion of reaction, the reaction mixture was cooled to 25° C. to 35° C., quenched with water (1000 ml) and extracted with ethylacetate (1000 ml). The organic phase was separated, dried on sodium sulfate and evaporated to give the title compound as brownish solid (68.0 g).

Yield: 85.0%

### Example 16

### Preparation of 1-[2-(2,4-Dimethyl-phenylsulfanyl)-phenyl]-4-methanesulfonyl-piperazine [Compound (VIIa) Wherein Pr is methanesulfonyl]

A solution of 1-[2-(2,4-Dimethyl-phenylsulfanyl)-phenyl]-4-methanesulfonyl piperazin-2-one (100 g, 0.256 mol) in Tetrahydrofuran (500 ml) was cooled at 0 to −10° C. 10M Borane-DMS solution (77.0 ml, 0.768 mol) was added to the reaction mixture and gradually heated at 50 to 60° C. for 4 to 5 h. Completion of the reaction was confirmed using thin layer chromatography (TLC). After completion of the reaction, the reaction mixture was cooled at 0° C. to 5° C. 10% aq. HCl solution (200 ml) was added to the reaction mixture and heated at 50-60° C. for 2 h. The reaction mixture was cooled at ambient temperature 25° C. to 35° C. and basified with 10% aq. NaOH solution till pH 10 to 12 was obtained. The reaction mixture was extracted in ethylacetate (500 ml). Organic phase was separated, washed with water (250 ml×2), dried over sodium sulfate and evaporated to give the title product (59.5 g).

Yield: 75.0%

### Example 17

### Preparation of 1-[2-(2,4-Dimethyl-phenylsulfanyl)-phenyl]-piperazine i.e. Vortioxetine (IX)

A solution of 1-[2-(2,4-Dimethyl-phenylsulfanyl)-phenyl]-4-methanesulfonyl-piperazine (100 g, 0.265 mol) in 30% HBr in acetic acid (500 ml) was heated at 50-60° C. for 10-12 h. Completion of the reaction was confirmed using thin layer chromatography (TLC). After completion of the reaction, the reaction mixture was cooled at 25-35° C. and water (500 ml) was added and stirred 1 h. The reaction mixture was filtered. The obtained solid was washed with water (50 ml×2) and suck dried. The wet solid was taken in separate flask and 20% aq. Sodium hydroxide solution was added to it till pH 10-12 was obtained. Ethylacetate (500 ml) was added to it and extracted. Organic phase was separated, washed with water (100 ml×2), dried over sodium sulfate and evaporated to give the title compound as white solid (80.2 g)

Yield: 80.

### Example 18

### Preparation of 1-[2-(2,4-dimethyl-phenylsulfanyl)-phenyl]-piperazin-2-one (VIII)

DIAD (83.25 g, 0.412 mol) was added dropwise to a solution of tri-n-butyl phosphine (92.30 g, 0.42 mol) in ethyl acetate (250 ml) keeping the temperature below 0° C. and continued stirring at the same temperature for 30 min to give yellow solution. In another flask, a solution of N-[2-(2,4-dimethyl-phenylsulfanyl)-phenyl]-2-(2-hydroxy-ethylamino)-acetamide (100 g, 0.302 mol) in ethyl acetate (600 ml) was cooled at 5° C. To this precooled solution, the above prepared yellow solution is added dropwise during 1 h maintaining the temperature below 5° C. The reaction mixture is brought to ambient temperature 25° C. to 35° C. over a period of 1 h and then heated to 40° C. to 50° C. for 2-3 h. Completion of reaction was confirmed using thin layer chromatography (TLC). Water was added to the reaction mixture and extracted. The organic phase was separated, dried and evaporated to give the title compound (80.4 g).

Yield: 85.0%

### Example 19

### Preparation of 2-[2-(2,4-Dimethyl-phenylsulfanyl)-phenylamino]-N-(2-hydroxy-ethyl)-acetamide (XI)

Aq.KHCO₃ solution (100.0 g, 1.000 mol, in 500 ml water) was added to a stirred solution of 2-(2,4-dimethyl-phenylsulfanyl)-phenylamine hydrochloride (100 g, 0.376 mol) in dichloromethane (1000 ml) over 10-20 minutes at 25-35° C. The reaction mixture was cooled at 0-10° C. 2-Chloro-N-(2-hydroxy-ethyl)-acetamide (69.82 g, 0.507 mole) was added to the reaction mixture and stirred at 0-10° C. for 1 to 2 h. Completion of reaction was confirmed using thin layer chromatography (TLC). After completion of the reaction, water (600 ml) was added to reaction mixture and extracted. The organic phase was separated, dried over sodium sulfate and evaporated to give the title product as off white solid (112.0 g).

Yield: 90%

### Example 20

### Preparation of [2-(2,4-dimethyl-phenylsulfanyl)-phenyl]-[(2-hydroxy-ethylcarbamoyl)-methyl]-carbamic acid tert-butyl Ester [Compound (p) Wherein Pr is Boc]

To a stirred mixture of 2-[2-(2,4-dimethyl-phenylsulfanyl)-phenylamino]-N-(2-hydroxy-ethyl)-acetamide (100 g, 0.302 mole), triethylamine (45.83 g, 0.453 mol) and dichloromethane (500 ml) was added Boc-anhydride (72.50 g, 0.332 mol) at 25-35° C. The reaction mixture was stirred at 25-35° C. for 2 to 3 h. Completion of reaction was confirmed using thin layer chromatography (TLC). After completion of the reaction, water (500 ml) was added to reaction mixture and extracted. Organic phase was separated, dried over sodium sulfate and evaporated to give the title product (110.0 g).

Yield: 85%

### Example 21

### Preparation of Methanesulfonic acid 2-(2-{tert-butoxycarbonyl-[2-(2,4-dimethyl-phenylsulfanyl)-phenyl]-amino}-acetylamino)-ethyl ester [Compound (q) wherein Pr is Boc; Lv is methanesulfonyl]

Methane sulphonyl chloride (65.10 g, 0.568 mol) was added to a precooled at 0-10° C. stirred mixture of [2-(2,4-dimethyl-phenylsulfanyl)-phenyl]-[(2-hydroxy-ethylcarbamoyl)-methyl]-carbamic acid tert-butyl ester (100 g, 0.232 mol), triethylamine (93.90 g, 0.928 mol) and dichloromethane (500 ml) at 0-10° C. and stirred for 2-3 hours at the same temperature. Completion of the reaction was confirmed using thin layer chromatography (TLC). After completion of the reaction, water (500 ml) was added to the reaction mixture and extracted. Organic phase was separated, dried over sodium sulfate and evaporated to give the title product (100.0 g).

Yield: 85%

### Example 22

### Preparation of Methanesulfonic acid 2-{2-[2-(2,4-dimethyl-phenylsulfanyl)-phenylamino]-acetylamino}-ethyl Ester [Compound (r) Wherein Lv is methanesulfonyl]

To a stirred solution of methanesulfonic acid 2-(2-{tert-butoxycarbonyl-[2-(2,4-dimethyl-phenylsulfanyl)-phenyl]-amino}-acetylamino)-ethyl ester (100 g, 0.196 mole) in ethyl acetate (250 ml) was added hydrochloric gas in isopropyl alcohol (0.392 mol, 14% w/v 95 ml) over 30 minutes at 25-35° C. and stirred further for 1 to 2 h. Completion of the reaction was confirmed using thin layer chromatography (TLC). After completion of the reaction, water (250 ml) was added to reaction mixture and adjusted pH of reaction mixture to 10 to 11 using 5% NaOH solution. Organic phase was separated, dried on sodium sulfate and evaporated to give the title compound (72.25 g)

Yield: 90%

### Example 23

### Preparation of 4-[2-(2,4-Dimethyl-phenylsulfanyl)-phenyl]-piperazin-2-one (XII)

A mixture of methanesulfonic acid 2-{2-[2-(2,4-dimethyl-phenylsulfanyl)-phenylamino]-acetylamino}-ethyl ester (100 g, 0.245 mol) in dimethyl formamide (500 ml) and K₂CO₃ (50.73 g, 0.367 mole) was heated at 50-60° C. for 10 to 12 h. Completion of the reaction was confirmed using thin layer chromatography (TLC). After completion of reaction, the reaction mixture was quenched with water (1000 ml) and extracted with ethyl acetate (1000 ml). The organic phase was separated, dried on sodium sulfate and evaporated to give the title compound (65.0 g). The crude compound was used for next step without further purification

Yield: 85%

### Example 24

### Preparation of 1-[2-(2,4-Dimethyl-phenylsulfanyl)-phenyl]-piperazine i.e. Vortioxetine (IX)

A solution of 4-[2-(2,4-dimethyl-phenylsulfanyl)-phenyl]-piperazin-2-one (100 g, 0.320 mol) in Tetrahydrofuran (500 ml) was cooled at 0 to −10° C. and stirred. 10M Borane-DMS solution (60.0 ml, 0.480 mol) was added to the above solution at the same temperature and stirred further for 4 to 5 h. The reaction progress was monitored on thin layer chromatography (TLC). After completion of the reaction, 10% aq. HCl solution (200 ml) was added to the reaction mixture and heated at 50-60° C. for 1 h. The reaction mixture was cooled and basified with 1N aq. NaOH solution till pH 10 to 11 obtained. The reaction mixture was extracted with ethyl acetate (500 ml). Organic phase was separated, dried over sodium sulfate and evaporated to give the title product (57.30 g). The crude product was used for next step without further purification.

Yield: 90.0%

### Example 26

### Preparation of 1-(2-Bromo-phenylsulfanyl)-2,4-dimethyl-benzene (XV)

A mixture of 1-Iodo-2,4-dimethylbenzene (100 g, 0.430 mol) and 2-bromobenzenethiol (81.45 g, 0.430 mol) in toluene (500 ml) was stirred with nitrogen gas purging at 25-35° C. In another flask, a mixture of Pd(dba)₂ (2.47 g, 0.0043 mol), Rac-BINAP (5.35 g, 0.0086 mol) and sodium tert butoxide (124.0 g, 1.29 mol) in toluene (250 ml) was stirred with nitrogen gas purging. This mixture was added to the above prepared mixture of 1-iodo-2,4-dimethylbenzene and 2-bromobenzenethiol and stirred for 2 to 3 h at 90-100° C. The reaction progress was monitored on thin layer chromatography (TLC). After completion of the reaction, the reaction mixture was cooled, quenched with water (500 ml) and extracted. The organic phase was separated and washed with water (500 ml), dried on sodium sulfate and evaporated to give the title product (82.10 g). The crude compound was used for next step without further purification.

Yield: 85.0%

### Example 27

### Preparation of 4-[2-(2,4-Dimethyl-phenylsulfanyl)-phenyl]-piperazin-2-one (XII)

A mixture of 1-(2-bromo-phenylsulfanyl)-2,4-dimethyl-benzene (100 g, 0.341 mol) and piperazine 2-one (85.26 g, 0.852 mol) in toluene (500 ml) was stirred with nitrogen gas purging at 25-35° C. In another flask, a mixture of Pd(dba)₂ (1.96 g, 0.0034 mol), Rac-BINAP (4.25 g, 0.0068 mol) and Sodium tert butoxide (98.23 g, 1.023 mol) in toluene (250 ml) was stirred with nitrogen gas purging. This mixture was added to the above prepared mixture of 1-(2-Bromo-phenylsulfanyl)-2,4-dimethyl-benzene and Piperazine 2-one and stirred for 4 to 5 h at 90-100° C. The reaction progress was monitored on thin layer chromatography (TLC). After completion of the reaction, the reaction mixture was cooled, quenched with water (500 ml) and extracted. The organic phase was separated and washed with water (500 ml), 1N HCl solution (500 ml), dried on sodium sulfate and evaporated to give the title product (58.57 g). The crude product was used for next step without further purification.

Yield: 80.0%

### Example 28

### Preparation of 2-Chloro-N-(2-hydroxy-ethyl)-acetamide (X)

To a stirred solution of ethanol amine (100 g, 1.64 mol) and triethylamine (332 g, 3.278 mol) in dichloromethane (500 ml) was added chloro acetyl chloride (203.8 g, 1.804 mol) at 0° C. to 10° C. and stirred further at the same temperature for 2-3 h. Completion of reaction was confirmed using thin layer chromatography (TLC). After completion of reaction, water (500 ml) was added to the reaction mixture and extracted. The organic phase was separated, dried on sodium sulfate and evaporated to give the title compound (192.0 g).

Yield: 85.0%

### Example 29

### Preparation of 2,4-Dimethyl-1-(2-nitro-phenylsulfanyl)-benzene (C)

Aq. NaOH solution (1.085 mol in 200 ml water) was added to a stirred solution of 2,4-dimethylbenzenethiol (100 g, 0.723 mol) in methanol (750 ml) at 25-35° C. 1-Chloro-2-nitro-benzene (136.83 g, 0.868 mol) was added to the reaction mixture and heated at 60-70° C. for 10 to 12 h. Completion of the reaction was confirmed using thin layer chromatography (TLC). After completion of the reaction, the reaction mixture was cooled at 25-35° C. and filtered. The solid obtained was washed with methanol (2×250 ml), suck dried and then dried in oven to give the title product as yellowish crystalline solid (159.4 g). The compound was used for next step without further purification.

Yield: 85.0%

### Example 30

### Preparation of 2-(2,4-dimethyl-phenylsulfanyl)-phenylamine hydrochloride (II)

To a stirred solution of 2,4-dimethyl-1-(2-nitro-phenylsulfanyl)-benzene (100 g, 0.386 mol) in glacial acetic acid (1000 ml) was added iron powder (86.40 g, 1.543 mol) at 25-35° C. and stirred. The reaction mixture was heated at 80-90° C. for 2 to 3 h. Completion of the reaction was confirmed using thin layer chromatography (TLC). After completion of the reaction, the reaction mixture was cooled at 25-35° C. and filtered through hyflo bed to remove salt. The hyflo bed was washed with ethyl acetate (600 ml). Combined filtrate was evaporated up to thick residue. The residue was neutralized using saturated NaHCO₃ solution and extracted with ethyl acetate (1000 ml). Organic phase was separated, dried over sodium sulfate and evaporated to give crude 2-(2,4-Dimethyl-phenylsulfanyl)-phenylamine as oil.

Hydrogen chloride gas in isopropyl alcohol (0.501 mol) was added over 30 minutes to a stirred solution of crude 2-(2,4-dimethyl-phenylsulfanyl)-phenylamine in mixture of ethyl acetate (240 ml) and cyclohexane (480 ml) at 25-35° C. The reaction mixture was stirred further at the same temperature for 1 to 2 h. Completion of reaction was confirmed using thin layer chromatography. After completion of the reaction, the reaction mixture was filtered. The solid obtained was washed with cyclohexane (2×100 ml), suck dried and dried in air oven to give the title product as white solid (74.75 g).

Yield: 84%

### Example 31

### Preparation of N-[2-(2,4-dimethyl-phenylsulfanyl)-phenyl]-2-(2-hydroxy-ethylamino)-acetamide (V)

Ethanol amine (91.62 g, 1.499 mol) was added to a stirred solution of 2-Chloro-N-[2-(2,4-dimethyl-phenylsulfanyl)-phenyl]-acetamide (100 g, 0.326 mol) in isopropyl acetate (800 ml) at 25-35° C. The reaction mixture was heated at 70-80° C. for 6 to 8 h. Completion of reaction was confirmed using thin layer chromatography (TLC). After completion of the reaction, the reaction mixture was cooled at 25-35° C. and quenched with water (1000 ml). The organic phase was separated, dried over sodium sulfate and evaporated to give crude product as oily mass. The crude product was purified over column chromatography using silica gel and mixture of ethyl acetate and cyclohexane as eluent to give pure title product as oil (70.0 g).

Yield: 90%

### Example 32

### Preparation of {[2-(2,4-dimethyl-phenylsulfanyl)-phenylcarbamoyl]-methyl}-(2-hydroxy-ethyl)-carbamic acid tert-butyl Ester (VI)

To a stirred mixture of N-[2-(2,4-dimethyl-phenylsulfanyl)-phenyl]-2-(2-hydroxy-ethylamino)-acetamide (100 g, 0.302 mol), triethylamine (45.83 g, 0.453 mol) and dichloromethane (500 ml) was added Boc-anhydride (72.50 g, 0.332 mol) at 25-35° C. The reaction mixture was stirred at 25-35° C. for 2 to 3 h. Completion of reaction was confirmed using thin layer chromatography (TLC). After completion of the reaction, water (500 ml) was added to reaction mixture and extracted. Organic phase was separated, dried over sodium sulfate and evaporated to give the title product as off white solid (91.14 g).

Yield: 90%

### Example 33

### Preparation of methanesulfonic acid 2-(tert-butoxycarbonyl-{[2-(2,4-dimethyl-phenylsulfanyl)-phenylcarbamoyl]-methyl}-amino)-ethyl Ester (VIb)

Methane sulphonyl chloride (65.10 g, 0.568 mol) was added to a precooled at 0-5° C. stirred mixture of {[2-(2,4-dimethyl-phenylsulfanyl)-phenylcarbamoyl]-methyl}-(2-hydroxy-ethyl)-carbamic acid tert-butyl ester (100 g, 0.232 mol), triethylamine (93.90 g, 0.928 mol) and dichloromethane (500 ml) at 0-10° C. and stirred for 2-3 hours at the same temperature. Completion of the reaction was confirmed using thin layer chromatography (TLC). After completion of the reaction, water (500 ml) was added to the reaction mixture and extracted. Organic phase was separated, dried over sodium sulfate and evaporated to give the title product as oily mass (59.0 g).

Yield: 93%

### Example 34

### Preparation of 4-[2-(2,4-dimethyl-phenylsulfanyl)-phenyl]-3-oxo-piperazine-1-carboxylic acid tert-butyl Ester (VII)

A mixture of methanesulfonic acid 2-(tert-butoxycarbonyl-{[2-(2,4-dimethyl-phenylsulfanyl)-phenylcarbamoyl]-methyl}-amino)-ethyl ester (100 g, 0.196 mol) in dimethyl formamide (500 ml) and K₂CO₃ (54.09 g, 0.392 mole) was heated at 50-60° C. for 10 to 12 h. Completion of the reaction was confirmed using thin layer chromatography (TLC). After completion of reaction, the reaction mixture was quenched with water (1000 ml) and extracted with ethyl acetate (1000 ml). The organic phase was separated, dried on sodium sulfate and evaporated to give the title compound as oily mass (52.65 g). The crude compound was used for next step without further purification

Yield: 87%

### Example 35

### Preparation of 1-[2-(2,4-dimethyl-phenylsulfanyl)-phenyl]-piperazin-2-one (VII)

To a stirred solution of 4-[2-(2,4-dimethyl-phenylsulfanyl)-phenyl]-3-oxo-piperazine-1-carboxylic acid tert-butyl ester (100 g, 0.242 mole) in ethyl acetate (250 ml) was added hydrochloric gas in isopropyl alcohol (0.363 mol, 14% w/v 95 ml) over 30 minutes at 25-35° C. and stirred further for 1 to 2 h. Completion of the reaction was confirmed using thin layer chromatography (TLC). After completion of the reaction, water (250 ml) was added to reaction mixture and adjusted pH of reaction mixture to 10 to 11 using 5% NaOH solution. Organic phase was separated, dried on sodium sulfate and evaporated to give the title compound as off white solid (53.0 g)

Yield: 96%

### Example 36

### Preparation of 1-[2-(2,4-dimethyl-phenylsulfanyl)-phenyl]-piperazine (IX)

A solution of 1-[2-(2,4-dimethyl-phenylsulfanyl)-phenyl]-piperazin-2-one (100 g, 0.320 mol) in Tetrahydrofuran (500 ml) was cooled at 0 to −10° C. 10M Borane-DMS solution (60.0 ml, 0.480 mol) was added to the reaction mixture and stirred at 0 to −10° C. for 4 to 5 h. Completion of the reaction was confirmed using thin layer chromatography (TLC). After completion of the reaction, 10% aq. HCl solution (200 ml) was added to the reaction mixture and heated at 50-60° C. for 1 h. The reaction mixture was cooled at ambient temperature and basified with 1N aq. NaOH solution till pH 10 to 11 obtained. The reaction mixture was extracted in ethylacetate (500 ml). Organic phase was separated, dried over sodium sulfate and evaporated to give the title product (59.20 g).

Yield: 88%

1. A process for preparation of vortioxetine hydrobromide (I) or its solvate 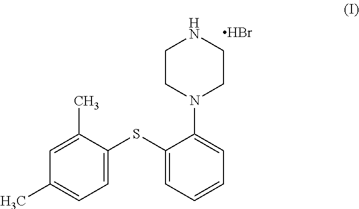 [SMILES: Br ; Cc1ccc(Sc2ccccc2N2CCNCC2)c(C)c1]
which comprises:
a) reacting 2-(2,4-dimethyl-phenylsulfanyl)-phenylamine (II) or its salt 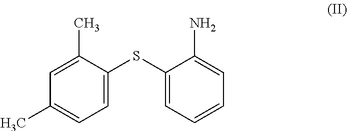 [SMILES: Cc1ccc(Sc2ccccc2N)c(C)c1]
with haloacetyl halide (III) 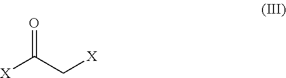
to give 2-halo-N-[2-(2,4-dimethyl-phenylsulfanyl)-phenyl]-acetamide (IV) 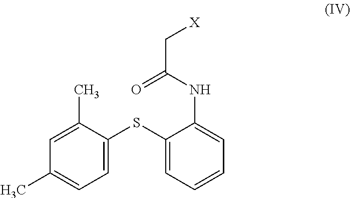
wherein X is halogen;
b) reacting 2-halo-N-[2-(2,4-dimethyl-phenylsulfanyl)-phenyl]-acetamide (IV) with ethanolamine or its salt to give N-[2-(2,4-dimethyl-phenylsulfanyl)-phenyl]-2-(2-hydroxy-ethylamino)-acetamide (V) or its salt; 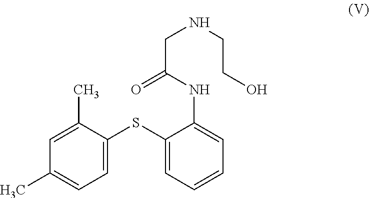 [SMILES: Cc1ccc(Sc2ccccc2NC(=O)CNCCO)c(C)c1]
c) reacting N-[2-(2,4-dimethyl-phenylsulfanyl)-phenyl]-2-(2-hydroxy-ethylamino)-acetamide (V) or its salt with a protecting reagent to give the compound of formula (VI) 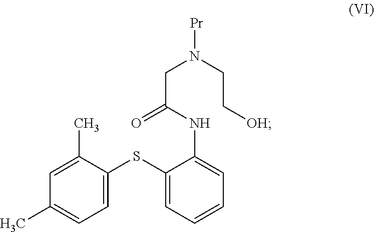
wherein Pr is a protecting group;
d) cyclizing the compound of formula (VI) to give the compound of formula (VII); 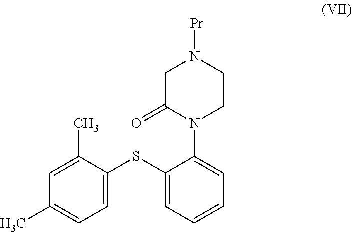
e) deprotecting protecting group Pr to give 1-[2-(2,4-dimethyl-phenylsulfanyl)-phenyl]-piperazin-2-one (VIII) or its salt; 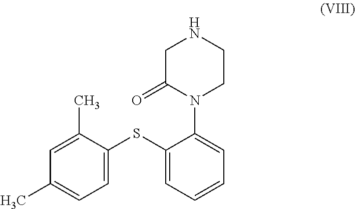 [SMILES: Cc1ccc(Sc2ccccc2N2CCNCC2=O)c(C)c1]
f) reducing 1-[2 (2,4-dimethyl-phenylsulfanyl)-phenyl]-piperazin-2-one (VIII) or its salt to give vortioxetine (IX) or its salt; 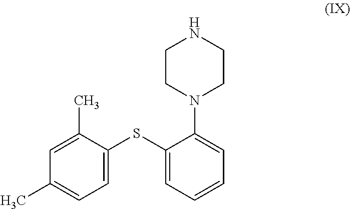 [SMILES: Cc1ccc(Sc2ccccc2N2CCNCC2)c(C)c1]
and converting vortioxetine (IX) or its salt to vortioxetine hydrobromide (I).

2. A process for preparation of vortioxetine or its salt or solvate comprising reducing the compound of formula (VIIIa):

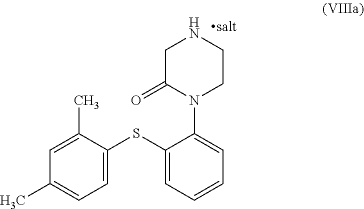 [SMILES: Cc1ccc(Sc2ccccc2N2CCNCC2=O)c(C)c1]

3. The process of claim 2, wherein the compound (VIIIa) is a hydrochloride salt having following formula:

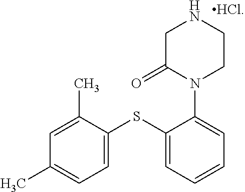

4. The process of claim 2, wherein reduction is carried out in presence of a reducing agent selected from lithium aluminum hydride (LiAlH₄); Sodium bis(2-methoxyethoxy) aluminumhydride (trade names Red-Al or vitride); Borane-dimethyl sulfide (Borane-DMS); a combination of borohydride and a lewis acid, wherein borohydride is selected from sodium borohydride (NaBH₄), lithium borohydride (LiBH₄), and sodium cyanoborohydride (NaCNBH₃) and the lewis acid is selected from ZnCl₂, AlCl₃, MgCl₂, BF₃ and TiCl₄; and a combination of borohydride and other reagent, wherein borohydride is selected from sodium borohydride (NaBH₄), lithium borohydride (LiBH₄), and sodium cyanoborohydride (NaCNBH₃) and the other reagent is selected from acetic acid, pyridine, POCl₃, trimethylsilyl.

5. The process of claim 1, wherein said protecting group Pr is selected from tertbutyloxycarbonyl (boc), triphenylmethyl (trityl), benzyloxycarbonyl (cbz), benzyl, trifluoroacetyl (COCF₃), acetyl, silyl or any other N-protecting or O-protecting group.

6. The process of claim 1, wherein step (c) further comprises preparing reactive derivative (VIb) from the compound of formula (VI) and cyclizing reactive derivative (VIb) to give the compound of formula (VII) 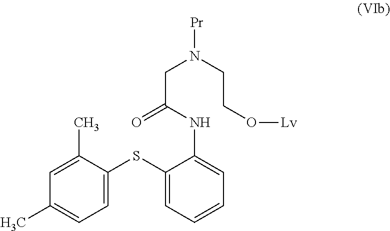
wherein Pr is protecting group and Lv is selected from tosyl, mesyl, and nosyl.

7. (canceled)

8. (canceled)

9. The process of claim 1 further comprising:
c) reacting N-[2-(2,4-dimethyl-phenylsulfanyl)-phenyl]-2-(2-hydroxy-ethylamino)-acetamide (V) or its salt with a protecting reagent to give the compound of formula (VIa); and 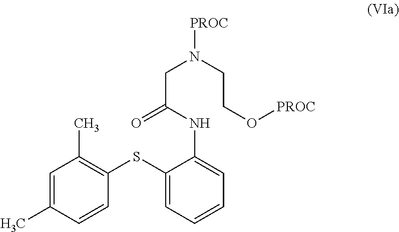
d) cyclizing the compound of formula (VIa) to give the compound of formula (VII); 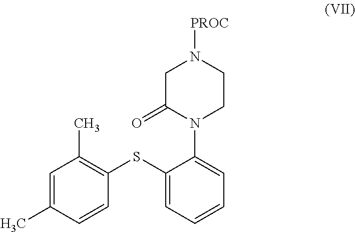
where in PROC is a protecting group which is so chosen that it acts as protecting group at a nitrogen atom and a leaving group at an oxygen atom when subjected to cyclization conditions.

10. (canceled)

11. (canceled)

12. The process of claim 9, wherein said protecting group PROC is selected from methane sulfonyl, p-toluene sulfonyl, nosyl, COCF₃ (trifluoro acetyl), acetyl, acyl, benzyl, substituted benzyl, benzoyl, trimethylsilyl, tert butyl dimethyl silyl (TBDMS), trifluoromethylsulfonate (OTf).

13. The process of claim 1, wherein step (e) deprotection is carried out by hydrolysis or hydrogenation depending on the protecting group.

14. The process of claim 1, wherein step (f) reduction is carried out in presence of reducing agent selected from lithium aluminum hydride (LiAlH₄); Sodium bis(2-methoxyethoxy) aluminumhydride (trade names Red-Al or vitride); Borane-dimethyl sulfide (Borane-DMS); a combination of borohydride and lewis acid, wherein borohydride is selected from sodium borohydride (NaBH₄), lithium borohydride (LiBH₄), sodium cyanoborohydride (NaCNBH₃) and the lewis acid is selected from ZnCl₂, AlCl₃, MgCl₂, BF₃, TiCl₄; and a combination of borohydride and other reagent, wherein borohydride is selected from sodium borohydride (NaBH₄), lithium borohydride (LiBH₄), and sodium cyanoborohydride (NaCNBH₃) and the other reagent is selected from acetic acid, pyridine, POCl₃, trimethylsilyl.

15. (canceled)

16. The process of claim 1 comprising:
e) deprotecting protecting group Pr from compound (VII) with acid to give a salt of 1-[2-(2,4-dimethyl-phenylsulfanyl)-phenyl]-piperazin-2-one (VIIIa); 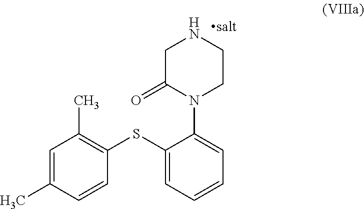 [SMILES: Cc1ccc(Sc2ccccc2N2CCNCC2=O)c(C)c1]
where in Pr is a protecting group;
f) reducing the salt of formula (VIIIa) to give a salt of vortioxetine (IXa); 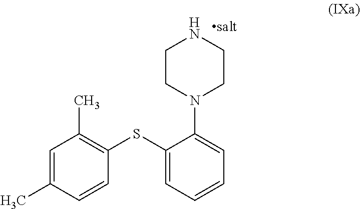 [SMILES: Cc1ccc(Sc2ccccc2N2CCNCC2)c(C)c1]
and converting salt of vortioxetine (IXa) to vortioxetine hydrobromide (I) or its solvate.

17. The process of claim 16, wherein the compound of formula (VIIIa) is a hydrochloride salt having following formula:

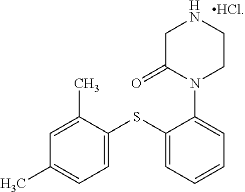

18. The process of claim 17, wherein reduction is carried out in presence of a reducing agent selected from lithium aluminum hydride (LiAlH₄); Sodium bis(2-methoxyethoxy) aluminumhydride (trade names Red-Al or vitride); Borane-dimethyl sulfide (Borane-DMS); a combination of borohydride and a lewis acid, wherein borohydride is selected from sodium borohydride (NaBH₄), lithium borohydride (LiBH₄), and sodium cyanoborohydride (NaCNBH₃) and the lewis acid is selected from ZnCl₂ AlCl₃, MgCl₂, BF₃, and TiCl₄; and a combination of borohydride and other reagent, wherein borohydride is selected from sodium borohydride (NaBH₄) lithium borohydride (LiBH₄), and sodium cyanoborohydride (NaCNBH₃) and the other reagent is selected from acetic acid, pyridine, POCl₃, trimethylsilyl.

19.-29. (canceled)

30. A compound which is selected from: 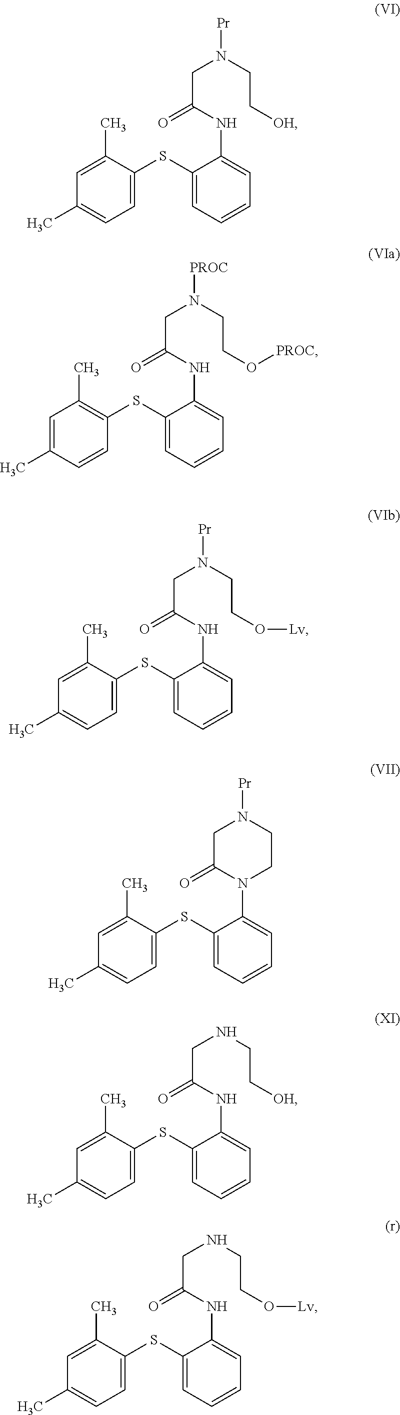 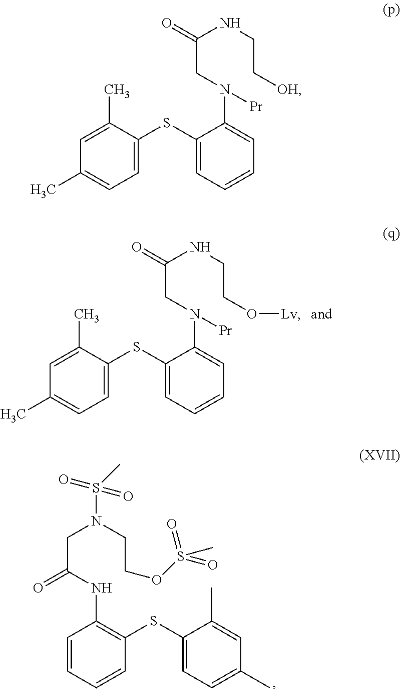
or a salt thereof;
wherein X is halogen; Pr is a protecting group; Lv is selected from mesyl, tosyl, and nosyl; salt is organic or inorganic salt; PROC is a protecting agent such that it act as protecting group at nitrogen and leaving group when attached to oxygen under cyclization condition.

31. A compound which is selected from: [chemical structure]
